(19) United States
(12) Patent Application Publication
(10) Pub. No.: US 2020/0193140 A1
(43) Pub. Date: Jun. 18, 2020

(54) Detection of Biological Cells or Biological Substances

(71) Applicant: Nano Global, Austin, TX (US)

(72) Inventors: Steven Papermaster, Austin, TX (US); Zoltan Papp, III, Austin, TX (US); Christine Scheve, Austin, TX (US); Aaron Papermaster, Austin, TX (US); Eric Crawford, Austin, TX (US)

(21) Appl. No.: 16/641,373

(22) PCT Filed: Aug. 22, 2018
(86) PCT No.: PCT/US2018/047568

§ 371 Date: Feb. 26, 2020

Related U.S. Application Data
(60) Provisional application No. 62/549,543, filed on Aug. 24, 2017.
(60) Provisional application No. 62/588,754, filed on Nov. 20, 2017.
(60) Provisional application No. 62/644,331, filed on Mar. 16, 2018.

(51) Int. Cl. G06K 9/00; A61L 2/24; A61L 2/16

(52) U.S. Cl. CPC G06K 9/00134; A61L 2/24; A61L 2202/16; A61L 2202/14; A61L 2/16

(57) ABSTRACT

Described herein are techniques related to the detection and/or identification of biological cells and/or substances. An example of an electronic device is described herein that includes a scene-capture system to obtain an image of a scene that includes cells or substances therein. The example electronic device also includes a biologic detection system to analyze the obtained image and detect and/or identify a biological cell and/or substance amongst the in-scene cells and/or substances and a reporting system to report a detection or identification of a type or class of biological cell and/or substance in the obtained image and/or identification of a particular cells and/or substances. This Abstract is submitted with the understanding that it will not be used to interpret or limit the scope or meaning of the claims.

### BACKGROUND

In biology, a cell is the basic structural, functional, and biological unit of all known living organisms. A cell is the smallest unit of life that can replicate independently, and cells are often called the “building blocks of life.” The term “cell” itself is very common. Herein, the term “biological cell” is used to distinguish the term from other common uses in other fields.

Typically, biological cells consist of cytoplasm enclosed within a membrane, which contains many biomolecules such as proteins and nucleic acids. Organisms can be classified as single-celled or unicellular (consisting of a single cell; including bacteria) or multicellular (such as plants and animals). While the multicellular plants and animals are often visible to the unaided human eye, their individual cells are visible only under a microscope, with dimensions between 1 and 100 micrometers.

Common examples of biological cells are microbes. Microbes (i.e., microscopic organisms) are microscopic living things that are found nearly everywhere on our planet. Indeed, macroscopic living things (e.g., humans) normally co-exist peacefully with microbes. Indeed, many microbes are helpful or essential to a healthy life and a healthy ecosystem.

Unfortunately, some microbes are harmful microorganisms. These are typically called pathogens. A bacterium is an example of a pathogen that may infect a human and produce disease. For example, listeria produces listeriosis and staphylococcus produces a staph infection.

Modern hygiene and sanitation techniques and technology have greatly reduced the chances of encountering pathogens. However, they have not eliminated the risk. Indeed, many people still fall ill or worse by coming in contact with pathogens. Often, these pathogens are transferred from one surface (e.g., countertop) to another (e.g., a hand) by surface contact.

Since microbes, by their nature, are too small to be seen by the unaided eye, a person is unaware of the relative cleanliness of a surface before touching that surface. Since all typical surfaces have some microbes thereon, a person is typically unable to know how much or what kind of microbes are on a surface.

### SUMMARY

Described herein are techniques related to the detection and/or ‘identification of biological cells and/or substances. An example of an electronic device is described herein that includes a scene-capture system to obtain an image of a scene that includes cells or substances therein. The example electronic device also includes a biologic detection system to analyze the obtained image and detect and/or identify a biological cell and/or substance amongst the in-scene cells and/or substances and a reporting system to report a of a type or class of biological cell and/or substance in the obtained image and/or identification of a particular cells and/or substances. This summary is submitted with the understanding that it will not be used to interpret or limit the scope or meaning of the claims.

### BRIEF DESCRIPTION OF THE DRAWINGS

FIG. 1 illustrates conventional techniques to detect and identify pathogens.

FIG. 2 illustrates an example system in accordance with the present disclosure.

FIG. 3 illustrates a chip architecture in accordance with the present disclosure.

FIG. 4 is a flow chart illustrating an example method in accordance with the present disclosure.

The Detailed Description references the accompanying figures. In the figures, the left-most digit(s) of a reference number identifies the figure in which the reference number first appears. The same numbers are used throughout the drawings to reference like features and components.

### DETAILED DESCRIPTION

Disclosed herein is technology to facilitate the detection and/or identification of pathobiological cells or substances. For example, with this technology, a potentially harmful pathogen can be detected and/or identified.

In some implementations, this detection and/or identification of pathobiological cells and/or substances are performed in situ (i.e., in place) and without any interference, contact, or adulteration by the tester or testing equipment. Thus, the technology described herein can facilitate the detection of and the nature of the threat of the spread of disease where and when the threat exists. With the in situ implementations, the detection and/or identification occurs in the field. That is, it does not rely on later and further testing at another location, thus it is in-the-field.

In addition, with the technology described herein, diseased cells (e.g., cancerous) can be detected and identified. In some implementations, the cells may be detected or identified on or inside a human body (which is an example of in situ) and without any interference, contact, or adulteration by the tester or testing equipment. Thus, the technology described herein can facilitate the detection of and the nature of the actual disease (e.g., cancer) where it hides on or in the human body. This detection does not rely on later and further testing at another location, thus it is in-the-field.

The detection and identification of pathogens (i.e., disease-causing) and pathological (i.e., diseased) cells is an important application of the technology described herein, but it is just only one application. More generally speaking, this technology may be used to study cells both in-the-field and in real time. More particularly, cells may be studied to determine what changes over time, discover correlations from those changes, and draw inferences from those correlations.

In this way, the cells being studied need not be pathobiological. They may be any cells. In particular, the subject cells may be ones where it would be desirable to study them in their natural environment rather than cultured in a lab, where it would be desirable to study them in real time. Herein, real-time involves cells life cycle in their normal course rather than cultured as they are typically done in a lab.

In other implementations of the technology described herein, disease-causing cells (e.g., pathogens) or disease-causing substances (e.g., toxins) may be detected or identified in situ or “in the lab.” As used herein, the term “in the lab” refers to the opposite of in situ. While the action may literally occur in a lab, the term more broadly refers to these actions (e.g., detection and identification) occurring not in place, not in the field, away from the location where the cells or substances are discovered, or with interference, contact, or adulteration by the tester or testing equipment. For example, growing collected cells in a petri dish occurs in the lab.

The hardware may include a system-on-a-chip (SoC) to be used in various devices, such as the Internet of Things (IoT) develops. The chip may be based on various architectures, such as RISC architectures, for example, Arm Limited based architectures. The chip can collect molecular data in real time, increasing the amount of available data by orders of magnitude.

An implementation using SoC may greatly reduce the data bandwidth required to be transmitted to the platform. Especially, when SoC is used for using object detection and identification processing.

### Example Scenario

Listeria monocytogenes is a pathogen that causes listeriosis, which is an infection with symptoms of fever, vomiting, and diarrhea. This pathogen is an example of a pathobiological cell. Listeria can spread to other parts of the body and lead to more serious complications, like meningitis. Listeria is often transmitted by ready-to-eat foods, such as milk, cheese, vegetables, raw and smoked fish, meat, ice cream, and cold cuts. This early and rapid detection is desirable so that cross-contamination can be avoided and any problems immediately addressed.

These ready-to-eat foods are often mass produced in food factories. In these factories, there is little to no time to stop production to test to determine if a harmful pathogen (like listeria) exists on the food-production surfaces. Depending on the comprehensiveness and desired accuracy of the test, conventional techniques to detect the bacteria take as long as a week to as short as hours. Regardless of the particulars of the test, these conventional tests involve the manual collection of samples from various surfaces, cataloging these samples, and performing invasive testing (e.g., culturing, chemical reaction, antibodies, etc.).

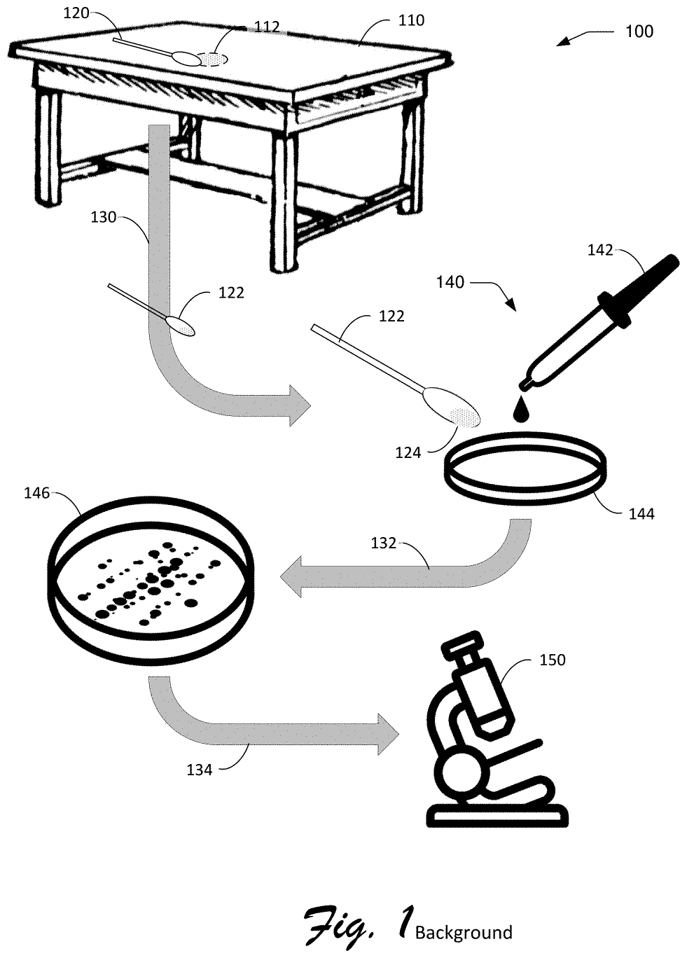

FIG. 1 illustrates conventional techniques 100 to detect and identify pathogens. Table 110 has a surface that, of course, has microbes thereon. This table 110 represents anything with a surface area that might have microbes living on it. For this discussion, assume that table 110 is in a commercial kitchen of a ready-to-eat food manufacturer. This manufacturer is concerned about Listeria in this kitchen. To detect the existence of Listeria in its kitchen, the manufacturer orders spot tests be performed in the kitchens.

To that end, a spot 112 on table 110 is selected for sampling. Using a sample-collection swab 120, a tester swipes the spot 112. Following arrow 130, a sample-collected swab 122 is carefully transported to a testing station 140 so as to avoid collateral and inadvertent collection of samples from other sources.

Typically, this testing station 140 is physically separated and distant from the sample spot 112 of the commercial kitchen where the sample was collected. The testing station 140 is often in a laboratory of a testing facility. With traditional methods, the sample microbes 124 of the sample-collected swab 122 are transferred to Petri dishes 144 for cultivation. At some point, chemicals 142 may be added to the cultured microbes of the Petri dishes 144 for various reasons, such as dyes to make them more visible.

Following arrows 132 and 134 and perhaps weeks or months, a petri dish 146 with the adultered (e.g., dyed) cultured microbes is ready to be examined under a microscope 150. Typically, a human examines the cultures under the microscope 150 and identifies pathogens amongst the cultured microbes based on many factors, but mostly the human's professional experience in identifying such microbes.

Traditional methods of testing like that demonstrated in FIG. 1, where sample microbes are cultivated in labs, are flawed. ‘Stressed’ cells will not grow in cultures (and will, therefore, produce negative results) despite the bacteria being present, live and potentially harmful. Also, this is the slowest form of testing.

Alternative conventional techniques, based on molecular/chemical methods, detect all cell types, but don't differentiate between live and harmless dead cells, which can remain after disinfection. Thus, these molecular/chemical methods may indicate a false positive for the presence of a pathogen when only dead cells of the pathogen are present.

Still, other conventional techniques use antibodies to test biofilms, which are groups of microbes where cells stick together on a surface. This technique requires the biofilms to be removed from the surface, treated with a particular antibody, and then tested to see if the biofilm fluoresces. This type of technique only tests for the particular pathogen that the introduced antibody interacts with.

### Example Electronic Device

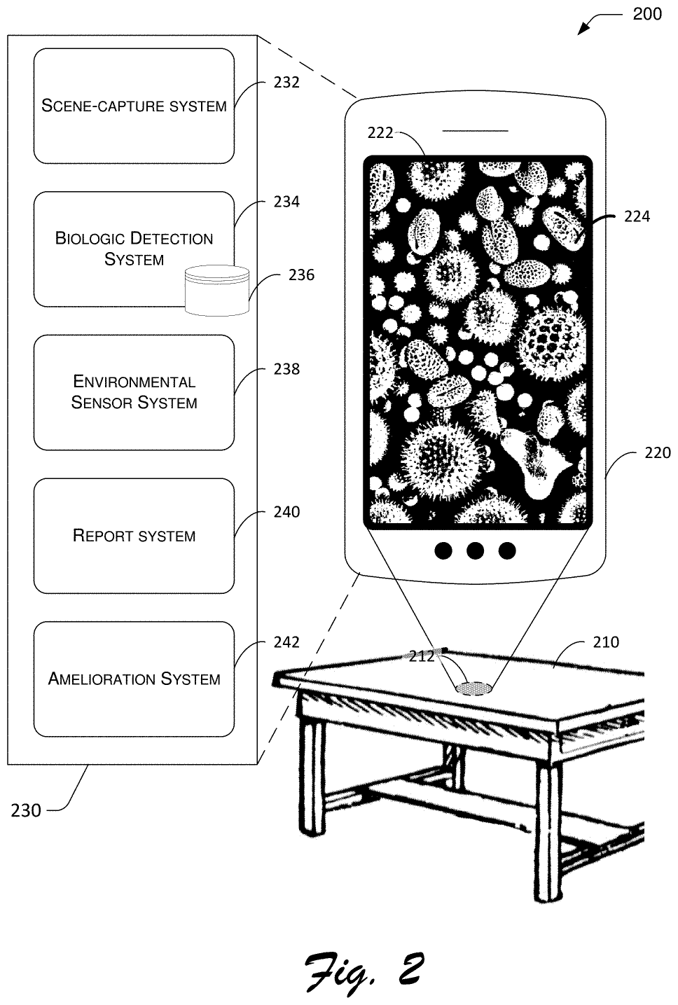

FIG. 2 illustrates an example scenario 200 in accordance with the technology described herein. The example scenario 200 includes a table 210. That table has a scene 212, which is an area of a surface in view of a camera (not shown) of a smartphone 220. Indeed, the camera captures an image 222 of scene 212. Just like reality, the scene 212 includes multiple microbes, but these microbes are not visible in scene 212.

For the example scenario 200, the microbes are described as in situ (i.e., in place) because they are examined, tested, etc. where they naturally live, inhabit, or exist. That is, the microbes are not in the lab. Herein, “in the lab” indicates that the microbes have been moved, relocated, or expatriated in order to perform the examination, testing, or the like. Other implementations of the technology described herein may involve microbes that are in the lab.

That image 222 is depicted as being viewed on a display of the smartphone 220. The image 222 has been sufficiently magnified to be able to see various in situ microbes of scene 212. And while not yet detected, one of these microbes is a pathogen 224.

The smartphone 220 is one example of an electronic device 230 in accordance with the technologies described herein. However, in other example scenarios, the electronic device 230 may be, for example, a tablet computer, a smartdevice, a standalone device, a collection of cooperative devices, a button-sized device, a device on a chip, an accessory to a smartphone or smartdevice, an ambulatory device, a robot, swallowable device, an injectable device, embedded within medical lab equipment, or the like.

As depicted, the electronic device 230 includes a scene-capture system, a biologic detection system (and object detection system) 234, a database 236, an environmental sensor system 238, a report system 240, and an amelioration system 242. These systems of the electronic device 230 are constructed from hardware, firmware, special-purpose components (e.g., sensors), and/or some combination thereof. These systems may, in some instances, include software modules as well.

The scene-capture system 232 is designed to obtain an image (e.g., image 222) of a scene (e.g., scene 212) that includes in situ biological cells therein. That is, there are biological cells located in a place (i.e., in-the-field) in the scene that is being captured by the scene-capture system. In some implementations, the scene-capture system 232 includes a camera to capture the visible part of the electromagnetic spectrum that is emitting or reflecting from the matter contained in the scene. In some implementations, the scene-capture system 232 includes components designed to capture non-visible parts of the electromagnetic spectrum (e.g., x-rays, infrared, gamma rays, ultraviolet, etc.) that is emitting or reflecting from the matter contained in the scene.

Examples of the action of obtaining (as performed by the scene-capture system 232) include measuring, collecting, accessing, capturing, procuring, acquiring, and observing. For example, the scene-capture system 232 may obtain the image by capturing the image using the charge-coupled device (CCD) of the digital camera. In another example, the scene-capturing system 232 may obtain the image by measuring the electromagnetic spectrum of the scene.

The obtained image is micrographic, spectrographic, digital, or some combination thereof. The obtained image is micrographic because it captures the elements in the scene that are on a microscopic scale. The obtained image is spectrographic because it captures elements in the scene by using equipment sensitive to portions of the electromagnetic spectrum (visible and/or non-visible portions). The obtained image is digital because it formats and stores the captured information as data capable of being stored in a machine, computer, digital electronic device, or the like.

While the thing that is captured is called an image, this image is not necessarily displayable as a two-dimensional depiction on a display screen (as shown in image 222). Rather, the image an array of data that represents the quantitative and qualitative nature of the electromagnetic spectrum (or some portion thereof) received by the components of the scene-capture system 232 when it was exposed to the scene (e.g., scene 212).

The biologic detection system 234 is designed to analyze the obtained image and detect the presence of one or more pathobiological cells amongst the in situ biological cells of the captured scene. In some implementations, the biologic detection system 234 may actually identify one or more particular cells and/or substances in the scene. In that case, it may be called a biologic identification system. Such a system may identify the particular pathobiological cells amongst the in situ biological cells. Thus, depending on the implementation, this system 234 may be referred to as biological-cell detection system or the pathobiological detection system.

To accomplish detection, the biologic detection system 234 may employ on and/or employ a database 236. This database 236 may be a database of pathobiologic-cellular signatures or training corpus. The biologic detection system 234 is a particular example of a biologic-cellular detection system. A training corpus is a database of numerous application-specific samples from which the AI/ML/DL engine “learns” and improves its capabilities and accuracy.

The biologic detection system 234 employs an AI/ML/DL engine to perform or assist in the performance of the detection and/or identification of one or more pathobiological cells. AI/ML/DL is short for artificial intelligence/machine learning/deep learning technology. Particular implementations may employ just an AI engine, just an ML engine, just a DL engine, or some combination thereof.

The AI/ML/DL engine may be implemented just on the smartphone 220 itself. In that case, the smartphone 220 need not communicate in real time with the platform (e.g., a remote computing system). In another implementation, the AI/ML/DL engine may be implemented just on the platform (thus remotely). In that case, the smartphone 220 communicates in real time (or nearly so) with the platform (e.g., a remote computing system). In still other implementations, the AI/ML/DL engine is implemented partially in both the smartphone 220 and the platform. In this way, the intensive processing is offloaded to the platform.

Some implementations of the biologic detection system 234 may perform its data analysis solely on the device without assistance from other devices, servers, or the cloud. Other implementations of the biologic detection system 234 may farm out all or nearly all of the data analysis to other devices, servers, or the cloud. In still other implementations, the data analysis may be shared amongst multiple devices and locations.

On its own or working with other devices or computer systems, the biologic detection system 234 analyzes the image of the scene to detect, determine, and/or identify the type or class of biological cells therein. It may do this, at least in part, by using distinguishing molecules of the cells that are observable using the electromagnetic spectrum. Is some implementations, other data (such as chemical reactions or excitation) may be included in the analysis.

Some of these molecules are indicative of certain classes, types, or particular cells. Such molecules are called marker biomolecules herein. The electronic device can determine which cell types or class are present in a captured scene by the on the particular ones of, the types of, and the proportions of the biomolecules detected therein. This may be accomplished, at least in part, by calculating probabilities of objects detected in the image.

The environmental sensor system 238 is designed to measure one or more environmental factors associated with the in situ biological cells or the environment surrounding the in situ biological cells. In some instances, the environmental sensor system 238 may be simply described as a sensor.

The report system 240 is designed to report detection and/or identification of the one or more pathobiological cells in the obtained image and in some implementations, the report system 240 is designed to associate the measured environmental factor with the obtained image and/or with the detected pathobiological cell.

The amelioration system 242 is designed to respond to the detection and/or identification in a manner that ameliorates the pathobiological nature of the detected/identified pathobiological cells.

The electronic device 230 may have a communications system to send or receive data from other similar electronic devices or centralized/distributed servers. The electronic device 230 may have enhanced processor or co-processor to perform image-capture and processing functions.

Of course, the example scenario 200 described above is one implementation that detects pathobiological cells. Other implementations of the technology described herein may detect or identify pathobiological substances rather than cells.

One or more of the systems of electronic device 230 may be characterized as a non-transitory computer-readable storage medium comprising instructions that when executed cause one or more processors of a computing device to perform the operations of that electronic device.

### Pre-Processed Data

Typically, images of the same scene are captured over time. That may be over a few seconds, few minutes, or perhaps a few hours. In so doing, a massive amount of raw image data is produced. So much data that it may quickly overwhelm the local storage capacity of the smartphone 220 and often overwhelms the data transfer rate between the smartphone 220 and any network-based storage solution.

To address this issue, the smartphone 220 maybe designed to pre-process or process the raw scene-captured data before storing locally or transferring it across the network. For pre-processing, the smartphone 220 may derive just the most critical or key data that helps identity or reconstruct the scene.

For example, the smartphone 220 may store the marker biomolecule information from each captured image or scene. The marker biomolecule information includes just the data regarding the type, amount, proportions, etc. of the marker biomolecules or substances detected, determined, identified, etc. in a particular image or scene. Thus, any other data from the image capture is discarded.

Along with associated environmental factors, this pre-processed information is stored or transferred across the network. This reduces the data storage/transfer requirements by multiple orders of magnitude. The particular cell type or class is determined from this stored or transferred pre-processed data. This may be done later or by different systems.

In some instances, the smartphone 220 may fully process the image/scene captured data to determine, detect, and/or identify the cell type or class. In this scenario, the electronic device stores or transfers its conclusion about the cell type or class with its associated environmental factors.

### Data Analysis

Since the images being captured are on a microscopic scale, it may take many images to capture a small surface area of an object. In addition, even a short sequence of images adds up quickly to a great multitude of images. Thus, in only a short space (e.g., of just a few seconds), the sequence of microscopic-scale images of a very small area quickly overwhelms the internal data transfer, data storage, and/or data processing capability of a typical electronic device (such as a smartphone). In addition, the typical external data transfer rates (e.g., of wireless communication) is not capable of accepting the data tsunami of this technology.

Two example approaches may be employed to address these issues. One involves the increased capacity of the electronic device, and the other involves the processing of the data into a manageable form.

First, this technology is implemented in such a way to employ special-purpose hardware to perform the pre-processing and processing of the incoming real-time data. That is, the specially designed hardware is built directly into the processors and electronics of the device to enable the device to quickly process the massive amount of incoming real-time data into a representative portion thereof without losing important aspects of the data.

Second, this technology employs a particular mechanism to produce a representative portion thereof without losing important aspects of the data. In short, that involves saving the deltas (i.e., changes) between the measurements (e.g., marker biomolecules) over time. These deltas are stored and/or transferred. In addition, data compression schemes may be used.

### Imaging

The technology described herein utilizes an image-capturing system, such as the scene-capture system 232. In some instances, the image-capturing system may be called a camera. This is particular so when the system captures the visible part of the electromagnetic spectrum that is emitting and/or reflecting from the matter being observed. In some implementations, the image-capturing system may capture non-visible parts of the electromagnetic spectrum (e.g., x-rays, gamma rays, ultraviolet, etc.) that are emitting or reflecting from the matter being observed.

With some implementations, the image-capturing system may employ hyperspectral imaging and, in particular, snapshot hyperspectral imaging. Hyperspectral imaging collects and processes information from across a portion of the electromagnetic spectrum. With hyperspectral imaging, the spectrum is captured for each pixel in the image of a scene. Snapshot hyperspectral imaging uses a staring array (rather than a scanning array) to generate an image in an instant.

With some implementations, the image-capturing system may employ light-field imaging, which is also called plenoptic imaging. A plenoptic imaging system captures information about the light field emanating from a scene. That is, it captures the intensity of light in a scene, and also the direction that the light rays are traveling in space. This contrasts with a conventional camera, which records only light intensity.

Using plenoptic imaging enables the simultaneous capture of pictures at different focal points, allowing the device sensor to capture a two-dimensional image in multiple 3rd dimension planes (i.e., capture a volume of space vs. a plane). Capturing a volume facilitates faster detection on non-flat surfaces or when fluids or gases are observed.

In addition, a combination of both these hyperspectral and plenoptic technologies may be used. That is, the image-capture system may incorporate both snapshot hyperspectral imaging with plenoptic imaging.

### Nanophotonics

In some instances, an agent is purposefully introduced into the scene, environment, or in the lab to enhance or improve the observations or measurements. For example, photonic nanostructures may be spread in the environment where measurements and observations may be made.

These photonic nanostructures are part of a field called nanophotonics or nano-optics, which involve is the study of the behavior of light on the nanometer scale, and of the interaction of nanometer-scale objects with light. It is a branch of optics, optical engineering, electrical engineering, and nanotechnology. It often (but not exclusively) involves metallic components, which can transport and focus light via surface plasmon polaritons.

The term “nano-optics,” just like the term “optics,” usually refers to situations involving ultraviolet, visible, and near-infrared light (free-space wavelengths from 300 to 1200 nanometers).

Using nanophotonics to create high peak intensities: If a given amount of light energy is squeezed into a smaller and smaller volume (“hot-spot”), the intensity in the hot-spot gets larger and larger. This is especially helpful in nonlinear optics; an example is surface-enhanced Raman scattering. It also allows sensitive spectroscopy measurements of even single molecules located in the hot-spot, unlike traditional spectroscopy methods which take an average over millions or billions of molecules.

One goal of nanophotonics is to construct a so-called “superlens”, which would use metamaterials or other techniques to create images that are more accurate than the diffraction limit (deep subwavelength).

Near-field scanning optical microscope (NSOM or SNOM) is another nanophotonic technique that accomplishes the same goal of taking images with resolution far smaller than the wavelength. It involves raster-scanning a very sharp tip or very small aperture over the surface to be imaged.

Near-field microscopy refers more generally to any technique using the near-field to achieve nanoscale, subwavelength resolution. For example, dual polarization interferometry has picometer resolution in the vertical plane above the waveguide surface.

### Environmental Factors

As indicated above, sensors obtain environmental factors related to, about, or near the scenes being observed. These may be called ambient factors. The sensors may measure or sense to obtain the environmental factors. In other instances, the factors may be accessed, acquired, procured, etc. via another source, sensor, memory, machine, etc.

The environmental factors are abiotic or biotic. However, there are other datapoints that may be gathered, but which are not expressly related to the environment. These may be called associated or observation-related factors.

An abiotic environmental factor is associated with non-biological sources. That is, the source of the thing being measured is not related to a living or recently living thing.

Examples of abiotic environmental factor include ambient temperature, timestamp (e.g., time and date), moisture, humidity, radiation, the amount of sunlight, and pH of a water medium (e.g., soil) where a biological cell lives. Other examples of abiotic environmental factors include barometric pressure; ambient sound; indoor location; ambient electromagnetic activity; velocity; acceleration; inertia; ambient lighting conditions; WiFi fingerprint; signal fingerprints; GPS location; geolocation; airborne particle counter; chemical detection; ; gases; radiation; air quality; airborne particulate matter (e.g., dust, 2.5 PPM, 10 PPM, etc.); atmospheric pressure; altitude; Geiger counter; proximity detection; magnetic sensor; rain gauge; seismometer; airflow; motion detection; ionization detection; gravity measurement; photoelectric sensor; piezo capacitive sensor; capacitance sensor; tilt sensor; angular momentum sensor; water-level (i.e., flood) detection; flame detector; smoke detector; force gauge; ambient electromagnetic sources; RFID detection; barcode reading; or some combination thereof.

A biotic environmental factor is one having a biologic source. Example of such include the availability of food organisms and the presence of conspecifics, competitors, predators, and parasites.

While it is not an environment factor, per se, the observation-related or associated factor is described here. The associated or observation-related factor may be a measurement of quality, quantity, and/or characteristic of the environment about the observation itself or related to the environment from which the subject is observed or was obtained. They may also be data that a human or computer associated with other environmental factors or the scene.

Examples of the observation-related or associated factor include nearby traffic patterns or noises; tracking the movements of particular individuals (e.g., via employee badges or security cameras); visitors; patients, budgets of related departments; and the like.

Herein, a known sensor or measurement device may be listed as an example of an environmental factor. For example, Geiger counter and seismometer are listed as examples. It should be understood that the relevant factor for least listed examples is the measurements typically made by such devices. Thus, the obtained factor for example Geiger counter is radiation and the obtained factor for the example seismometer is the motion of the ground.

### Artificial Intelligence, Machine Learning, and Deep Learning Technology

Herein, the term AL/ML/DL technology refers to a technology that employs known or new artificial intelligence (AI) techniques, machine learning (ML) techniques, deep learning (DL) techniques, and/or the like.

By applying AI/ML/DL technology such as convolutional neural networks (CNNs), some implementations of the technology described herein is capable of identifying pathobiological cells and substances within microscopic images and spectrographic signatures that environment and systems ingest from either existing datasets or streams of real-time sensor data.

By training its neural networks against libraries of high-quality pathobiological cells/substances images and signatures, the technology described herein can reliably identify specific cells/substances. Upon discovery, the technology described herein may take advantage of a sophisticated queueing system to retroactively “replay” historical data with a greatly increased sampling rate, enabling it to build a high-resolution model of the outbreak. This model is then added to the chain, fully secure, attributed and available to researchers who can use it to help contain the outbreak or to advance the understanding of its transmission model.

For example, the technology described herein can provide for real-time and real-world data. The chip is used in the data ingest pipeline and marketplace. Deployed throughout a building and/or across a region and using the sensor technology to pick up environmental (e.g. temperature, humidity, etc.), visible, and spectrographic data which, coupled with ambient other (e.g., location, time, etc.) data, the numerous chips in the system can together stream enormous volumes of valuable data into the platform for processing by the artificial intelligence insights engine described herein. As used herein, a platform includes a remote device with massive storage capacity and processing power.

The technology described herein may utilize AI to detect objects that have already been learned by a device or platform implementing the technology. This minimizes the amount of data transmitted, efficiently utilizing communication bandwidth. The sensed and other data associated with objects that the technology described herein detects, but cannot identify, will be sent to the platform which would, in turn, trigger an investigation that gathers real-world samples and take those samples to a lab for controlled analysis and identification using machine learning with deep learning. Once identified in the lab, the platform can send a specific detection approach to an implementation of the technology described herein so that it can then confidently identify the new object is going forward.

The technology described herein will either contain or connect to sensors that will enable novel abilities to detect pathobiological cells and substances at low concentrations, in noisy environments (e.g. objects in the midst of dust, dirt, or other uninteresting objects), in real-time (e.g. without significant delay from when object was in field of view), in some cases without disturbing the environment detected objects (i.e. passive observation), and with the ability to search large three dimensional spaces so as to reduce the probability of not observing an interesting object which is especially important when the objects are infrequently present.

Having some or all of these qualities, coupled with detection assisted by AI/ML/DL engines will facilitate detection and identification that is much faster than present technology, more accurate, and possible outside of a lab environment.

Some implementations of the technology described herein utilize AL/ML/DL technology in the detection or identification of pathobiological cells or substances from collected data. In other implementations, the technology descried herein may utilize AL/ML/DL technology in the analysis of the collected data with metadata such as environmental factors collected therewith.

According to Apr. 4, 2018, cloudmayo.com article (“Difference between Artificial Intelligence, Machine Learning and Deep Learning”), there is much confusion between three different but interrelated technologies of AI, ML, and DL. The article defines AI as a “technique that allows a computer to mimic human behavior,” ML as a “subset of AI techniques that uses a statistical method to enable a machine to improve with experiences,” and DL as a “subset of ML which makes the computation of multi-layer neural networks feasible.”

The electronic component or collection of components that employs an AI/ML/DL technology for training and/or data analysis is called an AI/ML/DL technology herein.

### System-on-a-Chip

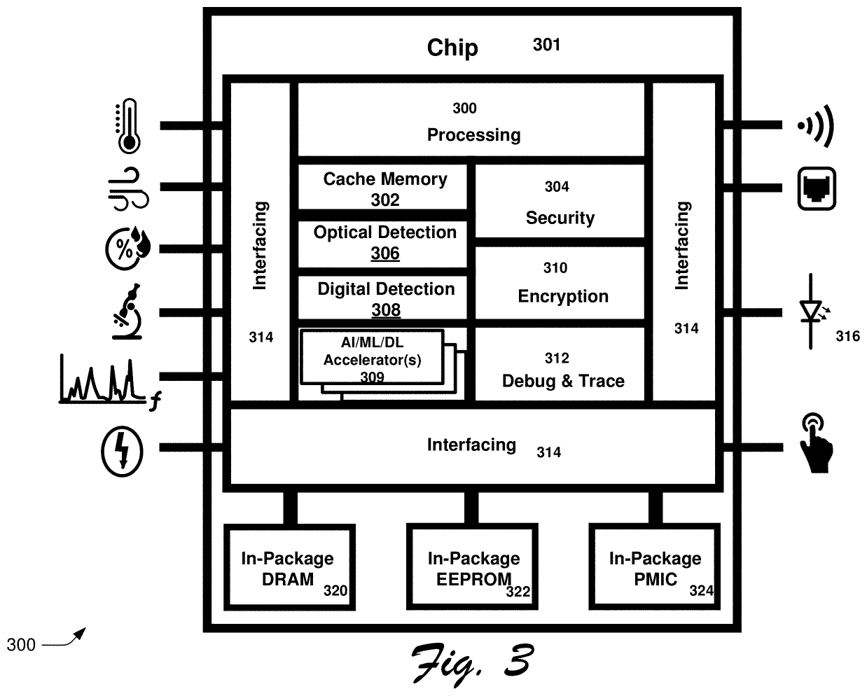

FIG. 3 illustrates an example system-on-a-chip 400, which is an implementation of the technology described herein. As shown, the system-on-a-chip 400 include a semiconductor chip integrated into a single package or as a chipset 301. Although a particular chip architecture is described, it is to be understood that other semiconductor chip architectures may be implemented.

The chip 301 can be resident in different devices, such as smart phones, laptop/table computers, dedicated medical/research devices, etc. Such devices are used for detection of pathobiological cells or substances. In certain implementations, computing may be performed off-chip via the cloud, or performed on the chip.

The chip 301, and in particular devices including the chip 301, may use AI/ML/DL engines to process data. In particular, AI/ML/DL engines may be used by the chip 301 in the accelerated processing of collected data, such as environmental factors and other data to detect/identify pathobiological cells and substances. In addition, doing so reduces data bandwidth of communication (e.g., to the platform). Also, distributed processing at the device reduces the cost of the device and reduces communication bottlenecks.

The chip 301 includes a processor(s) or processing component 300, cache memory 302, security component 304, optical detection 306, and digital detection 308. Depending on the implementation, the digital detection 308 may be used for one or more of the following: digital enhancement, object detection, or object identification.

Chip 301 can include one or more AI/ML/DL engines or accelerators 309. The AI/ML/DL accelerators 309 can implement edge and/or cloud computing. Chip 301 further can include encryption 310, and debug and trace component 312.

Interfaces 314 are provided/included in chip 301. In particular interfaces 314 provide the ability to receive sensory input, such as environmental factors (e.g., temperature, air pressure, wireless signals, capacitance, etc.) and capture image of a scene (e.g., via a microscope, camera, or spectrometer). The interface 314 also allows for transmission of data, network connections, user input, status indicators, and control illumination circuitry.

Interfaces 314 may further connect, for example, to an in-package dynamic random-access memory (DRAM) 320, in-package electrically erasable programmable read-only memory (EEPROM) 322, and power management integrated circuit (PMIC) 324.

### Example Processes

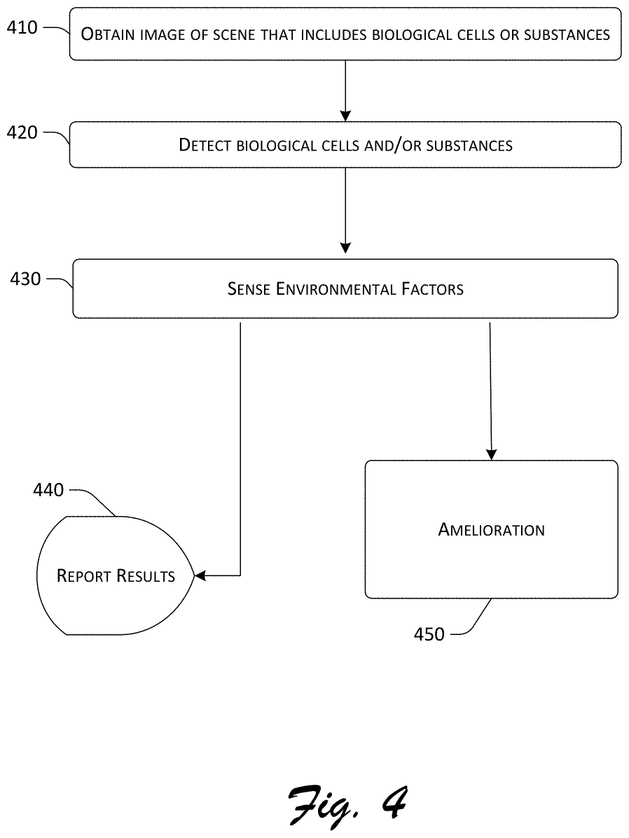

FIG. 4 is a flow diagram illustrating example process 400 that implement the techniques described herein for the detection and/or identification of biological cells and/or biological substances. For example, the example process 400 may detect and/or identify pathobiological cells and/or substances.

The example process 400 may be performed, at least in part, by the electronic device 230 and/or by the system-on-a-chip 301 as described herein. The example process 400 may be implemented by other electronic devices, computers, computer systems, networks, and the like. For illustration purposes, the example process 400 is described herein as being performed by a “device.”

At block 410, the device obtains an image of a scene that is the user presumes to include biological cells and/or substances therein. Indeed, the device may obtain a sequence of images of the scene. If any particular type or class of biological cells and/or substances are detected, then the scene included biological cells and/or substances.

A scene may include, for example, one or more surfaces on which the in-scene biological cells and/or substances inhabit; a liquid in which the in-scene biological cells and/or substances inhabit; a bodily fluid in which the in-scene biological cells and/or substances inhabit; an area in which the in-scene biological cells and/or substances inhabit; a volume in which the in-scene biological cells and/or substances inhabit; an area or volume with its dimensions falling below 0.1 mm; or a combination thereof.

In some implementations, the scene includes biologic cells or biologic substances. But, often, the scene includes both. The in-scene biological cells and/or substances may be characterized as: physically located on a surface; physically located in a medium (e.g., blood, bodily fluids, water, air, etc.); undisturbed in their environment; undisturbed and unadulterated; physically located on a surface in a manner that is undisturbed and unadulterated; not relocated for the purpose of image capture; unmanipulated for the purpose of image capture; or on a surface that is unaffected by the scene-capture system.

In some implementations, the biologic cells and/or substances are in situ and in other implementations, they are in the lab.

The obtained image is micrographic, spectrographic, and/or digital. In some implementations, the obtained image is micrographic because the image of the scene is captured at least in part: on a microscopic scale; using microscope like magnification; includes microscopic structures and features; includes structures and features that are not visible to a naked human eye; or a combination thereof.

In some implementations, the obtained image is spectrographic at least in part because the image of the scene is captured using some portion of the electromagnetic spectrum (e.g., visible spectrum of light, infrared, x-rays, gamma rays, ultraviolet) as it interacts with matter, such interactions include, for example, absorption, emission, scattering, reflection, and refraction.

The image may be obtained by capturing a digital image of the scene, and that scene may include in-scene biological cells and/or substances therein. In addition, digital enhancement of a captured digital image may be employed to better reveal the in-scene biological cells and/or substances in the captured image. The obtained image is digital at least in part because the image of the scene has handled as a set of machine-readable data.

With the electronic device 230, for example, the scene-capture system 232 performs this operation. The scene-capture system may be characterized as: a camera; a digital camera; a still image camera; a video camera; a digital camera with micro-optics that optical magnification for obtaining the image; a digital camera with micro-optics are part of the electronic device itself; a digital camera with micro-optics are part of a smartdevice (e.g., smartphone or tablet computer) with its own processor and camera (as its scene-capture system); a digital camera with micro-optics are part of additional camera functionality provided by an accessory or case for a smartdevice that operatively attaches to the smartdevice and adds the additional processing capabilities and functionalities for its scene-capture system; a flush mounted bead (i.e., lens) to perform optical magnification for its scene-capture system; a 12 (or greater) megapixel camera for its scene-capture system; a 12 (or greater) megapixel camera with flush mounted micro-optic bead (i.e., lens) to do optical magnification for its scene-capture system; a sapphire lens for its scene-capture system; captures an image using at least some portion of the visible electromagnetic spectrum; captures an image using at least some portion of the non-visible electromagnetic spectrum; captures an image using infrared light waves; captures an image using ultraviolet light waves; captures an image using ambient in-environment electromagnetic sources; captures images using ambient in-environment electromagnetic sources only; captures an image using artificial visible lighting which is added to the environment via artificial lighting sources associated with the scene-capture system; captures an image using artificial non-visible lighting which is added to the environment via artificial non-visible lighting sources associated with the scene-capture system; captures the image of the scene with magnification; captures the image of the scene with an magnification of a range of 20-200×; captures the image of the scene with an magnification of a range of 200-1000×; captures the image of the scene with an magnification of a range of 1000-5000×; captures the image of the scene with an magnification of a range of 2500-5000×; captures the image of the scene with an magnification of a range of 5000-10000×; captures the image of the scene with an magnification of greater than 1000×; captures the image of the scene with an magnification of greater than 5000×; captures a sequence of magnified images of the scene; enhances the image of the scene with digital magnification; enhances the image of the scene with digital image enhancement (e.g., sharpness, contrast, brightness, hue adjustment, etc.); enhances the image of the scene to a magnification level achievable by an electron scanning microscope (ESM); or a combination thereof.

At block 420, the device analyzes the obtained image and detects a type or class of biological cells or substance amongst the in-scene biological cells and/or substance. For example, the type or class of detected biological cell and/or substance may be a pathobiological cell and/or substance.

Examples of one or more of the types or classes of biological cells that may be detected and/or identified at block 420 includes (by way of example, but not limitation): cells of a multicell biological organism; cells of a tissue or organ of a multicell biological organism; cells of a tumor or growth multicell biological organism; single-celled organism; microbes; microscopic organisms; single-celled organism; living things that are too small to be seen with a human's naked eye; a biological creature that can only be seen by a human with mechanical magnification; microscopic spores; a combination thereof. In addition, the biological cells have a size range that is selected from a group consisting of: 10-100 nanometers (nm); 10-80 nm; 10-18 nm; 15-25 nm; and 50-150 nm.

Furthermore, biological cells and/or substances may be typed or classified as pathobiological, not pathobiological, pathobiology unknown, or pathobiology not-yet-known. The pathobiological biological cells and/or substances may be classified or typed as (by way of example and not limitation): pathobiological cells; pathobiological substances; toxic; poisonous; carcinogenic; diseased cells; cancer cells; infectious agents; pathogens; bioagents; disease-producing agents; or combination thereof.

Of those biological cells that are characterized as microbes, they may be further typed or classified as one or more of the following (by way of example and not limitation): single-celled organisms; bacteria; archaea; fungi; mold; protists; viruses; microscopic multi-celled organisms; algae; bioagents; spores; germs; prions; a combination thereof.

In some instances, the operation at block 420 may include an identification of one or more particular biologic cells and/or substances in the scene. Rather than just detecting the type or class (e.g., pathogen), the operation may identify the member of that type or class (listeria). Examples of particular members of a class or type that this operation may identify include: clostridium botulinum, streptococcus pneumoniae, mycobacterium tuberculosis, escherichia coli o157:h7, staphylococcus aureus, vibrio cholerae, ebola, hiv, influenza virus, norovirus, zika virus, aspergillus spp, and entamoeba histolytica.

At block 420, one or more implementations of the detection and/or identification includes operations to:
access a database of signatures of biological cells and/or substances;
isolate a biological cell and/or substance in the obtained image;
correlate the isolated biological cell and/or substance to at least one signature in the database;
determine that the correlation is significant enough to indicate a sufficient degree of confidence to identify the isolated biological cell and/or substance as being a biological cell and/or substance;
in response to that correlation determination, label the isolated biological cell and/or substance as being the determined biological cell and/or substance.

An example of a “sufficient degree of confidence” includes more likely than not. A confidence factor for the “sufficient degree of confidence” may be weighted relative to a perceived degree of threat. For example, a pathogen that is unlikely to cause a human infection may have a very high confidence factor (e.g., 80%). Thus, a detection may only be noted if it is at least 80% likely to be that particular pathogen. Conversely, a pathogen may be particularly dangerous (e.g., small pox) and have only a small confidence factor (e.g., 20%). In this way, the dangerous pathogen is detected even it is more likely that the pathogen was misdetected.

Other implementations of the detection and/or identification include operations to:
provide the obtained image to a trained biological detection/identification (detection and/or identification) engine, the trained biological detection/identification engine being an AI/ML/DL engine trained to detect and/or identify biological cells and/or substances based on a training corpus of signatures of biological cells and/or substances;
receive a positive indication from the biological detection/identification engine that the obtained image includes a biological cell and/or substance therein and/or the identity of that biological cell and/or substance.

Still other implementations of the detection and/or identification include operations to:
provide the obtained image to a trained biological detection/identification engine, the trained biological detection/identification engine being an AI/ML/DL engine trained to detect and/or identify pathobiological cells and/or substances based on a training corpus of signatures of pathobiological cells and/or substances;
receive a positive indication from the biological detection/identification engine that the obtained image includes a pathobiological cell and/or substance therein and/or the identity of that pathobiological cell and/or substance,
wherein the obtained image includes data captured from the visible and/or non-visible electromagnetic spectrum of the scene.

Other variations of the detection and/or identification operations described above may be focused pathobiological cells and/or substances in particular.

At block 430, the device measures one or more environmental factors associated with the in-scene biological cells and/or substances or the environment surrounding the in-scene biological cells and/or substances. In some implementations, the device may acquire information related to or associated with the scene. In the electronic device 230, the environmental sensor system 238 performs this operation.

The measured environmental factors include (but are not limited to): temperature; timestamp (e.g., time and date, local time, incremental time, etc.), humidity; barometric pressure; ambient sound; location; ambient electromagnetic activity; ambient lighting conditions; WiFi fingerprint; signal fingerprints; GPS location; airborne particle or chemical detector/counter; gases; radiation; air quality; atmospheric pressure; altitude; Geiger counter; proximity detection; magnetic sensor; rain gauge; seismometer; airflow; motion detection; ionization detection; gravity measurement; photoelectric sensor; piezo capacitive sensor; capacitance sensor; tilt sensor; angular momentum sensor; water-level (i.e., flood) detection; flame detector; smoke detector; force gauge; ambient electromagnetic sources; RFID detection; barcode reading; or a combination thereof.

At block 440, the device reports a detection and/or identification of the type of biological cell and/or substances in the obtained image. In the electronic device 230, the report system 240 performs this operation. For example, the device may provide a report or notification via a user interface to a user. A messaging system (e.g., email or SMS) may be used for such notification.

In some implementations, the device may report that the type of biological cell and/or substances in the obtained image is a category flagged for further research and inquiry. For example, the device may be unable to detect the type of cell or substance. In that case, the device flags this as a something worthy of further inquiry. This category may be the default when there is a failure to detect or identify a cell or substance. In some instances, this category is only triggered with particular markers (e.g., chemicals or structures) are detected.

The report or notification may include the following (by way of example and not limitation) operations: send a communication (e.g., message, postal mail, email, text message, SMS message, electronic message, etc.) to a human or machine that is designated to receive such communications via wired or wireless communications mechanisms; send a notification (e.g., message, postal mail, email, text message, SMS message, electronic message, push notices, etc.) to a human or machine that is designated to receive such notification via wired or wireless communications mechanisms; update a database designated to receive such updates via wired or wireless communications mechanisms; store in memory (e.g., local or remote) the detection; or a combination thereof.

In addition, at block 440, the device associates the measured environmental factor and/or the associated factor with the obtained image and/or with the detected type or class of biological cell and/or substance. This association may perform in one more database. Indeed, such a database may take the form of a distributed ledger (DL) technology.

At block 460, the device initiates a reaction to the detection and/or identification of particular classes or types in a manner that neutralizes the harmful effects of the detected and/or identified biological cells and/or substances. In the case of the detection/identification of pathobiological cells and/or substances, the device ameliorates the pathobiological nature of the detected/identified pathobiological cell and/or substance.

In the electronic device 230, the amelioration system 242 performs this operation. For example, the amelioration actions may include (by way of example and not limitation):
introducing an active material (e.g., sanitizer, ultraviolet light, cleaning fluid/spray) to a physical location of the pathobiological cell and/or substance to neutralize (e.g., clean, reduce, kill, or eliminate) the pathobiological nature of the detected/identified pathobiological cell and/or substance;
dispatch or request a visit by a robot or human to a physical location of the pathobiological cell and/or substance to neutralize (e.g., clean, reduce, kill, or eliminate) the pathobiological nature of the detected/identified pathobiological cell and/or substance;
dispatch or request a visit by a robot or human to a physical location of the pathobiological cell and/or substance to document (e.g., photograph and measure) the conditions around the pathobiological cell and/or substance (e.g., a macroscopic photograph of the physical location);
activate an operation of a proximate electronic device or system that is proximate a physical location of the pathobiological cell and/or substance to neutralize (e.g., clean, reduce, kill, or eliminate) the pathobiological nature of the detected/identified pathobiological cell and/or substance;
activate an operation of a proximate camera that is proximate a physical location of the pathobiological cell and/or substance to document (e.g., photograph and video) the area around the pathobiological cell and/or substance; or
a combination thereof.

In some instances, the amelioration operation may include mitigation. For example, a recommendation may be made and/or directions given to institute treatment for an allergy if mold is present, increase the frequency of cancer check-ups, reallocate cleaning resources, etc.

### Glossary

The following is a list of relevant terms used herein. Unless the context in which the term is used indicates differently, the terms of this glossary may be understood as being described in this glossary in accordance with the technology described herein.

Electronic Device: An apparatus that includes one or more electronic components designed to control or regulate the flow of electrical currents for the purpose of information processing or system control. An electronic device may include some mechanical, optical, and/or otherwise non-electronic parts in addition to its electronic components. Examples of such electronic components include transistors, diodes, capacitors, integrated circuits, and the like. Often such devices have one or more processors that are capable of executing instructions, memories, input/output mechanisms (e.g., display screens, touchscreens, cameras, etc.), and communication systems (e.g., wireless networking and cellular telephony). Examples of an electronic device contemplated herein includes a smartphone, a tablet computer, medical equipment, a microscope, a smartdevice, a computer, a standalone unit, a collection of cooperative units, a button-sized unit, system-on-a-chip, a device on a chip, an accessory to a smartphone or smartdevice, an ambulatory device, a robot, swallowability device, an injectable device, or the like. Depending on the implementation, the electronic device may be characterized as: portable; handheld; fits into a typical pocket; lightweight; portable and with fixed (non-aimable) optics—thus, the device must be moved to aim the optics; with aimable optics—thus, the device need not be moved to aim the optics; or a combination thereof. In addition, an implementation of an electronic device may be characterized as a smartdevice (e.g., smartphone or tablet computer) with its own processor and camera (as its scene-capture system); an accessory or case for a smartdevice that operatively attaches to the smartdevice and adds additionally processing capabilities and functionalities for its scene-capture system; stand-alone device with its own processor and camera (as its scene-capture system); ambulatory device that can move under its own power; a device-on-a-chip; system-on-a-chip; or a wireless device that is configured to interconnect with a wireless network of such devices, this device has its own processor camera (as its scene-capture system).

System: An assemblage or combination of things, parts, or components that form a unitary or cooperative whole. In some instances, a system and platform are used synonymously.

Scene: An area, place, location, scenario, etc. that is in view of the scene-capture system.

Image: An array (e.g., two-dimensional) of data derived from and mapped to a scene. An image may be an array of measured data regarding the electromagnetic spectrum emanating from, reflected off, passing through, scattering off of, etc. the contents (e.g., matter) of the scene. The image has an inherent frame or bound around or surrounding the subject scene.

In situ: Describes something that is situated in the original, natural, or existing place or position. Something that is in place or position. It is undisturbed.

In-the-field: A synonym for in situ.

In the lab: Describes the opposite of in situ. That is, it describes something that has been removed from its original or natural place or position. It is something that is not in place. It has been repositioned.

Biological cell: In biology, a cell is the basic structural, functional, and biological unit of all known living organisms. Typically, biological cells consist of cytoplasm enclosed within a membrane, which contains many biomolecules such as proteins and nucleic acids. Organisms can be classified as single-celled or unicellular (consisting of a single cell; including bacteria) or multicellular (such as plants and animals). While the multicellular plants and animals are often visible to the unaided human eye, their individual cells are visible only under a microscope, with dimensions between 1 and 100 micrometers.

Biological substance: As used herein, a biological substance is not itself a biological cell. Rather, this is a substance is strongly associated with biological cells or lifeforms. In particular, a biological substance maybe part of or produced by a biological cell or lifeform. In other instances, a biological substance is capable of affecting a lifeform (or some portion thereof).

Biological cells and/or substances: As used herein, this refers to both “biological cells” and “biological substances.”

Type or class of biological cell: The cells may be classified, categorized, or typed based on identifiable characteristics (e.g., physical, chemical, behavioral, etc.). For example, some cells may be classified as pathological because they cause disease. Some may be a diseased typed because they are malfunctioning and/or infected.

Micrographic: An image is classified as micrographic when it captures content that is on a microscopic scale. Such content includes things that are less than 100 micrometers in size. More generally, it includes items smaller than a macroscopic scale (which are visible to the unaided human eye) and quantum scale (i.e., atomic and subatomic particles).

Spectrographic: An image is classified as spectrographic when it captures the interaction between matter and some portion of the electromagnetic spectrum. Examples of such interactions included absorption, emission, scattering, reflection, refraction, translucency, etc.

Optical: Physics that involves the behavior and properties of light, including its interactions with matter and instruments that use or detect it. However, optics involve more than just the visible spectrum.

Visible Spectrum: This is part of the spectrographic image but specifically includes some portion of the visible spectrum (i.e., light) and excludes the non-visible portions.

Digital: This describes data that is formatted and arranged so as to be managed and stored by a machine, computer, digital electronic device, or the like. A data in the form of a digital signal uses discrete steps to transfer information.

Disease: Any disordered or malfunctioning lifeform or some portion thereof. A diseased lifeform is still alive but ill, sick, ailing, or the like.

Pathological: Something that is capable of causing disease or malfunction in a lifeform (or a portion thereof). A pathogen is pathological.

Pathobiological: Something is pathobiological if it is either capable of causing the disease to a lifeform (or some portion thereof) or is a diseased lifeform (or some portion thereof).

Pathobiological cell: A biological cell that is pathobiological.

Pathobiological substance: This is a substance that is either capable of causing the disease to a lifeform (or some portion thereof) or is associated with a diseased lifeform (or some portion thereof). The substance is not itself a biological cell.

Pathobiological cells and/or substances: As used herein, the term “pathobiological” modifies both “cell” and “substance.”

Pathogen: A biological cell (e.g., unicellular organism) that is capable of causing a disease. More generally, anything that can cause or produce disease.

Diseased cell: A biological cell (e.g., cancerous cell) that is alive but diseased.

Lifeform: The body form that characterizes an organism. Examples of lifeforms include:
Plants—Multicellular, photosynthetic eukaryotes
Animals—Multicellular, eukaryotic organisms
Fungus—Eukaryotic organisms that include microorganisms such as yeasts and molds
Protists—a Eukaryotic organism that is not an animal, plant or fungus
Archaea—Single-celled microorganisms
Bacteria—Prokaryotic microorganisms

Organism: An organism may generally be characterized as containing different levels of the organization; utilizing energy; responding to stimuli/environment; maintaining homeostasis; undergoing metabolism; growing; reproduction; and adapting to its environment.

Environmental factor: It is anything measurable that is capable of affecting the scene or that is associated with the scene. Such things can be abiotic or biotic. Abiotic factors include, for example, ambient temperature, moisture, humidity, radiation, the amount of sunlight, and pH of the water medium (e.g., soil) where a microbe lives. Examples of biotic factors include the availability of food organisms and the presence of conspecifics, competitors, predators, and parasites.

Smartphone: Generally, this term refers to a portable electronic device with features that are useful for mobile communication and computing usage. Such features the ability to place and receive voice/video calls, create and receive text messages, an event calendar, a media player, video games, GPS navigation, digital camera and video camera.

Smartdevice: The concept of a smartdevice includes a smartphone, but it also includes any other portable electronic device that might not have all of the features and functionality of a smartphone. Examples of a smartdevice include a tablet computer, portable digital assistant, smart watches, fitness tracker, location trackers, a so-called internet-of-things device, and the like. A smartdevice is an electronic device that is generally connected to other devices or networks via different wireless protocols such as Bluetooth, NFC, Wi-Fi, 3G, etc., that can operate to some extent interactively and autonomously.

Accessory: As used herein, this is an accessory to an electronic device (such as a smartphone or smartdevice). It adds additional functionality and/or capabilities to the electronic device. Examples of such accessories include a smartwatch or electronically enabled phone case.

### Other Applications

In addition to the example scenarios and applications discussed above, the following are other example scenarios and applications in which the technology described herein may be employed. Of course, there are still other scenarios and applications in which the technology described herein may be employed, but they are not listed here.

Hospital cleanliness: Using a handheld device, the surfaces and equipment may be regularly checked to confirm their cleanliest and alert for the need to redouble sanitation/cleanliness procedures. Other forms of a device (e.g., robot, mounted device, etc.) may be used for the same purposes.

In-room monitoring: Using one or more of small wireless communicating devices, critical areas may be continuously monitored for any dangers (e.g., pathogens). For example, a fixed device may be placed in the HVAC ducting of a building to monitor the presence of potentially harmful bacteria or allergens in the ventilation system. In another example, an ambulatory device (e.g., robot) may travel around a room looking for potentially infectious agents.

Application of sanitizers and cleaners: A robotic version of the electronic device may be particularly suited for both detecting potentially dangerous pathogens and neutralizing the threat by delivering sanitizing and/or cleaning agents to an area inhabited by the dangerous pathogens.

On-person monitoring: Using a device-on-a-chip approach, a person may discretely wear a device designed to monitor the surfaces and liquids that the person encounters each day. Alternatively, the device may be attached to the person herself to monitor her skin or anything on the skin.

In vivo monitoring: A highly miniaturized device may be injected into the bloodstream of an animal or human. The device may passively flow with the blood, or it may have its own propulsion system. This device seeks out diseased cells (e.g., cancer) in the bloodstream or in tissues accessible therefrom.

Application of medicine: A version of the device may be placed on or in a living body to respond to the detection of diseased cells by delivering medicine designed to eliminate that disease.

### Additional and Alternative Implementation Notes

With some implementations, the technology is anchored by a platform. Depending on the implementation, a platform may be a system or device that includes just hardware (e.g., semiconductor chips, printed circuit boards, enclosure, etc.) or just firmware or some combination thereof. In other implementations, the platform may include a combination of software with hardware, firmware, or both.

While many implementations of the technology described herein are directed towards actions directed at in situ subjects (e.g., pathobiological cells and substances), some implementations may involve “in the lab” conditions. That is, the subject of the actions of these implementations are not necessarily in situ or undisturbed. Indeed, the subject may be repositioned, relocated, adjusted, adulterated, etc. The subject of these implementations may be handled in the traditional manner, such as microbes may be cultured in a petri dish. In this case, these implementations may be incorporated into or be an accessory to traditional data gathering equipment, such as a microscope.

In the above description of example implementations, for purposes of explanation, specific numbers, materials configurations, and other details are set forth in order to better explain the present disclosure. However, it will be apparent to one skilled in the art that the subject matter of the claims may be practiced using different details than the examples ones described herein. In other instances, well-known features are omitted or simplified to clarify the description of the example implementations.

The terms “techniques” or “technologies” may refer to one or more devices, apparatuses, systems, methods, articles of manufacture, and/or executable instructions as indicated by the context described herein.

As used in this application, the term “or” is intended to mean an inclusive “or” rather than an exclusive “or.” That is, unless specified otherwise or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more,” unless specified otherwise or clear from context to be directed to a singular form.

These processes are illustrated as a collection of blocks in a logical flow graph, which represents a sequence of operations that may be implemented in mechanics alone, with hardware, and/or with hardware in combination with firmware or software. In the context of software/firmware, the blocks represent instructions stored on one or more non-transitory computer-readable storage media that, when executed by one or more processors or controllers, perform the recited operations.

Note that the order in which the processes are described is not intended to be construed as a limitation, and any number of the described process blocks can be combined in any order to implement the processes or an alternate process. Additionally, individual blocks may be deleted from the processes without departing from the spirit and scope of the subject matter described herein.

The term “computer-readable media” is non-transitory computer-storage media or non-transitory computer-readable storage media. For example, computer-storage media or computer-readable storage media may include, but are not limited to, magnetic storage devices (e.g., hard disk, floppy disk, and magnetic strips), optical disks (e.g., compact disk (CD) and digital versatile disk (DVD)), smart cards, flash memory devices (e.g., thumb drive, stick, key drive, and SD cards), and volatile and non-volatile memory (e.g., random access memory (RAM), read-only memory (ROM)).

The following are examples of implementations of the technology described herein:

Example 1: An electronic device comprising: a scene-capture system to obtain an image of a scene that includes biological cells and/or substances therein; a biologic detection system to analyze the obtained image and detect a type or class of biological cells and/or substance amongst the in-scene biological cells and/or substances; a report system to report detection of the type of biological cells and/or substances in the obtained image.

Example 2: An electronic device of Example 1, wherein the type or class of detected biological cell and/or substance is a pathobiological cell and/or substance.

Example 3: An electronic device of Example 2, wherein the pathobiological biological cells includes: pathologic cells; diseased cells; cancer cells; infectious agents; pathogens; bioagents; disease-producing agents; combination thereof.

Example 4: An electronic device of Example 1, wherein the in-scene biological cells and/or substances are in situ.

Example 5: An electronic device of Example 1, wherein the obtained image is micrographic, spectrographic, and/or digital.

Example 6: An electronic device of Example 5, wherein the obtained image is micrographic because the image of the scene is captured by the scene-capture system at least in part: on a microscopic scale; using a microscope like magnification; includes microscopic structures and features; includes structures and features that are not visible to a naked human eye; or a combination thereof.

Example 7: An electronic device of Example 5, wherein the obtained image is spectrographic at least in part because the scene-capture system captures the image of the scene using some portion of the electromagnetic spectrum as it interacts with matter, such interactions include, for example, absorption, emission, scattering, reflection, and refraction.

Example 8: An electronic device of Example 1, wherein the obtained image is hyperspectral and/or plenoptic.

Example 9: An electronic device of Example 1, wherein the scene-capture system employs a near-field scanning optical microscope to obtain the image.

Example 10: An electronic device of Example 1, wherein the in-scene biological cells and/or substances include biological cells and/or substances that are characterized as: physically located on a surface; physically located in a medium; undisturbed in their environment; undisturbed and unadulterated; physically located on a surface in a manner that is undisturbed and unadulterated; not relocated for the purpose of image capture; unmanipulated for the purpose of image capture; or on a surface that is unaffected by the scene-capture system.

Example 11: An electronic device of Example 1, wherein the biological cells are characterized as: cells of a multicell biological organism; cells of a tissue or organ of a multicell biological organism; cells of a tumor or growth multicell biological organism; single-celled organism; microbes; microscopic organisms; single-celled organism; living things that are too small to be seen with a human's naked eye; a biological creature that can only be seen by a human with mechanical magnification; microscopic spores; or a combination thereof.

Example 12: An electronic device of Example 1, wherein the biological cells are characterized as microbes that are characterized as: single-celled organisms; bacteria; archaea; fungi; mold; protists; viruses; microscopic multi-celled organisms; algae; bioagents; spores; germs; prions; or a combination thereof.

Example 13: An electronic device of Example 1, wherein the biological cells have a size range that is selected from a group consisting of: 10-100 nanometers (nm); 10-80 nm; 10-18 nm; 15-25 nm; and 50-150 nm.

Example 14: An electronic device of Example 1, wherein the scene includes: one or more surfaces on which the in-scene biological cells and/or substances inhabit; a liquid in which the in-scene biological cells and/or substances inhabit; a bodily fluid in which the in-scene biological cells and/or substances inhabit; an area in which the in-scene biological cells and/or substances inhabit; a volume in which the in-scene biological cells and/or substances inhabit; an area or volume with its dimensions falling below 0.1 mm; or a combination thereof.

Example 15: An electronic device of Example 1, wherein the scene-capture system to obtain a sequence of images of the scene.

Example 16: An electronic device of Example 1, wherein the scene-capture system is characterized as: a camera; a digital camera; a still image camera; a video camera; a digital camera with micro-optics that optical magnification for obtaining the image; a digital camera with micro-optics are part of the electronic device itself; a digital camera with micro-optics are part of a smartdevice with its own processor and camera; a digital camera with micro-optics are part of additional camera functionality provided by an accessory or case for a smartdevice that operatively attaches to the smartdevice and adds the additional processing capabilities and functionalities for its scene-capture system; a flush mounted bead lens to perform optical magnification for its scene-capture system; a 12 or greater megapixel camera for its scene-capture system; a 12 or greater megapixel camera with flush mounted micro-optic bead lens to do optical magnification for its scene-capture system; a sapphire lens for its scene-capture system; captures an image using at least some portion of the visible electromagnetic spectrum; captures an image using at least some portion of the non-visible electromagnetic spectrum; captures an image using infrared light waves; captures an image using ultraviolet light waves; captures an image using ambient in-environment electromagnetic sources; captures images using ambient in-environment electromagnetic sources only; captures an image using artificial visible lighting which is added to the environment via artificial lighting sources associated with the scene-capture system; captures an image using artificial non-visible lighting which is added to the environment via artificial non-visible lighting sources associated with the scene-capture system; captures the image of the scene with optical magnification; captures the image of the scene with an optical magnification of a range of 20-200×; captures the image of the scene with an optical magnification of a range of 200-1000×; captures the image of the scene with an optical magnification of a range of 1000-5000×; captures the image of the scene with an optical magnification of a range of 2500-5000×; captures the image of the scene with an optical magnification of a range of 5000-10000×; captures the image of the scene with an optical magnification of greater than 1000×; captures the image of the scene with an optical magnification of greater than 5000×; captures a sequence of magnified images of the scene; enhances the image of the scene with digital magnification; enhances the image of the scene with digital image enhancement; enhances the image of the scene to a magnification level achievable by an electron scanning microscope (ESM); a combination thereof.

Example 17: An electronic device of Example 1 further comprising an environmental sensor system to obtain an environmental factor associated with the in-scene biological cells and/or substances or the environment surrounding the in-scene biological cells and/or substances.

Example 18: An electronic device of Example 17, wherein the obtained environmental factors includes biotic, abiotic, and associated factors.

Example 19: An electronic device of Example 17, wherein the environmental sensor system obtains the environmental factor by measurement or sensing.

Example 20: An electronic device of Example 17, wherein the report system to associate the obtained environmental factor with the obtained image and/or with the detected type or class of biological cell and/or substance.

Example 21: An electronic device of Example 17, wherein the obtained environmental factors are selected from a group consisting of: temperature; timestamp; humidity; barometric pressure; ambient sound; location; ambient electromagnetic activity; ambient lighting conditions; WiFi fingerprint; GPS location; airborne particle counter; chemical detection; gases; radiation; air quality; airborne particulate matter; atmospheric pressure; altitude; Geiger counter; proximity detection; magnetic sensor; rain gauge; seismometer; airflow; motion detection; ionization detection; gravity measurement; photoelectric sensor; piezo capacitive sensor; capacitance sensor; tilt sensor; angular momentum sensor; water-level detection; flame detector; smoke detector; force gauge; ambient electromagnetic sources; RFID detection; barcode reading; or a combination thereof.

Example 22: An electronic device of Example 1, wherein the detection of the biologic system includes operations to: access a database of signatures of biological cells and/or substances; isolate a biological cell and/or substance in the obtained image; correlate the isolated biological cell and/or substance to at least one signature in the database; determine that the correlation is significant enough to indicate a sufficient degree of confidence to identify the isolated biological cell and/or substance as being a biological cell and/or substance; in response to that correlation determination, label the isolated biological cell and/or substance as being the determined biological cell and/or sub stance.

Example 23: An electronic device of Example 1, wherein the detection of the biologic detection system includes operations to: provide the obtained image to a trained biological detection engine, the trained biological detection engine being an AI/ML/DL engine trained to detect and/or identify biological cells and/or substances based on a training corpus of signatures of biological cells and/or substances; receive a positive indication from the biological detection engine that the obtained image includes a biological cell and/or substance therein and/or identity of that biological cell and/or sub stance.

Example 24: An electronic device of Example 1, wherein the detection of the biologic detection system includes operations to: provide the obtained image to a trained biological detection engine, the trained biological detection engine being an AI/ML/DL engine trained to detect and/or identify biological cells and/or substances based on a training corpus of signatures of biological cells and/or substances; receive a positive indication from the biological detection engine that the obtained image includes a biological cell and/or substance therein and/or identity of that biological cell and/or substance, wherein the obtained image includes data captured from the visible and/or non-visible electromagnetic spectrum of the scene.

Example 25: An electronic device of Example 1, wherein the report system to report an identification of the biological cell and/or substance in the obtained image.

Example 26: An electronic device of Example 1, wherein the report of the report system is characterized by performing operations that: send a communication to a human or machine that is designated to receive such communications via wired or wireless communications mechanisms; send a notification to a human or machine that is designated to receive such notification via wired or wireless communications mechanisms; update a database designated to receive such updates via wired or wireless communications mechanisms; store the detection in a memory; a combination thereof.

Example 27: An electronic device of Example 1, wherein the report of the report system indicates that the type of biological cells and/or substances in the obtained image is a category flagged for further research and inquiry.

Example 28: An electronic device of Example 1 further comprising an amelioration system to react to the detection and/or identification in a manner that ameliorates the neutralizes harmful effects of the detected and/or identified biological cells and/or substances.

Example 29: An electronic device of Example 1 further comprising an amelioration system to react to the detection and/or identification in a manner that ameliorates the neutralizes harmful effects of the detected and/or identified biological cells and/or substances, wherein the amelioration action is characterized as: introducing an active material to a physical location of the biological cell and/or substance to neutralize the biological nature of the detected/identified biological cell and/or substance; dispatch or request a visit by a robot or human to a physical location of the biological cell and/or substance to neutralize the biological nature of the detected/identified biological cell and/or substance; dispatch or request a visit by a robot or human to a physical location of the biological cell and/or substance to document the conditions around the biological cell and/or substance; activate an operation of a proximate electronic device or system that is proximate a physical location of the biological cell and/or substance to neutralize the biological nature of the detected/identified biological cell and/or substance; activate an operation of a proximate camera that is proximate a physical location of the biological cell and/or substance to document the area around the biological cell and/or substance; a combination thereof.

Example 30: An electronic device of Example 1, wherein, in whole or in part, the electronic device is selected from a group consisting of: a system-on-a-chip; a device-on-a-chip; a smartdevice; a computer; an ambulatory device; a microscope; a mobile device; and a wireless device.

Example 31: An electronic device of Example 1, wherein the biologic detection system transmits the obtained image or a portion thereof across a communications network to a remote computer system.

Example 32: An electronic device of Example 1, wherein the scene includes photonic nanostructures therein.

Example 33: A method comprising: obtaining an image of a scene that includes biological cells and/or substances therein, wherein the obtained image is micrographic, spectrographic, and/or digital; analyzing the obtained image and detecting a type or class of biological cells and/or substance amongst the in-scene biological cells and/or substances; reporting detection of the type of biological cells and/or substances in the obtained image.

Example 34: A method of Example 33, wherein the type or class of detected biological cell and/or substance is a pathobiological cell and/or substance.

Example 35: A method of Example 33, wherein the in-scene biological cells and/or substances are in situ.

Example 36: A method of Example 33 further comprising: sensing an environmental factor associated with the in-scene biological cells and/or substances or the environment surrounding the in-scene biological cells and/or substances; associating the obtained environmental factor with the obtained image and/or with the detected type or class of biological cell and/or substance.

Example 37: A method of Example 33, wherein the detection operation includes: providing the obtained image to a trained biological detection engine, the trained biological detection engine being an AI/ML/DL engine trained to detect and/or identify biological cells and/or substances based on a training corpus of signatures of biological cells and/or substances; receiving a positive indication from the biological detection engine that the obtained image includes a biological cell and/or substance therein and/or identity of that biological cell and/or substance.

Example 38: A method of Example 33, wherein detection operation includes: providing the obtained image to a trained biological detection engine, the trained biological detection engine being an AI/ML/DL engine trained to detect and/or identify biological cells and/or substances based on a training corpus of signatures of biological cells and/or substances; receiving a positive indication from the biological detection engine that the obtained image includes a biological cell and/or substance therein and/or identity of that biological cell and/or substance, wherein the obtained image includes data captured from the visible and/or non-visible electromagnetic spectrum of the scene.

Example 39: A non-transitory computer-readable storage medium comprising instructions that when executed cause a processor of a computing device to perform operations comprising: obtaining an image of a scene that includes biological cells and/or substances therein, wherein the obtained image is micrographic, spectrographic, and/or digital; analyzing the obtained image and detecting a type or class of biological cells and/or substance amongst the in-scene biological cells and/or substances; reporting detection of the type of biological cells and/or substances in the obtained image.

Example 40: A non-transitory computer-readable storage medium of Example 39, wherein the type or class of detected biological cell and/or substance is a pathobiological cell and/or substance.

Example 41: A non-transitory computer-readable storage medium of Example 39, wherein the in-scene biological cells and/or substances are in situ.

Example 42: A non-transitory computer-readable storage medium of Example 39, wherein the operations further comprise: sensing an environmental factor associated with the in-scene biological cells and/or substances or the environment surrounding the in-scene biological cells and/or substances; associating the obtained environmental factor with the obtained image and/or with the detected type or class of biological cell and/or substance.

Example 43: A non-transitory computer-readable storage medium of Example 39, wherein the detection operation includes: providing the obtained image to a trained biological detection engine, the trained biological detection engine being an AI/ML/DL engine trained to detect and/or identify biological cells and/or substances based on a training corpus of signatures of biological cells and/or substances; receiving a positive indication from the biological detection engine that the obtained image includes a biological cell and/or substance therein and/or identity of that biological cell and/or sub stance.

Example 44: A non-transitory computer-readable storage medium of Example 39, wherein the detection operation includes: providing the obtained image to a trained biological detection engine, the trained biological detection engine being an AI/ML/DL engine trained to detect and/or identify biological cells and/or substances based on a training corpus of signatures of biological cells and/or substances; receiving a positive indication from the biological detection engine that the obtained image includes a biological cell and/or substance therein and/or identity of that biological cell and/or substance, wherein the obtained image includes data captured from the visible and/or non-visible electromagnetic spectrum of the scene.

Example 45: An electronic device comprising: a scene-capture system to obtain an image of a scene that includes in situ biological cells and/or substances therein, wherein the obtained image is micrographic, spectrographic, and/or digital; a biologic detection system to analyze the obtained image and detect pathobiological cells and/or substance amongst the in-scene biological cells and/or substances; a report system to report a detection of the pathobiological cells and/or substances in the obtained image.

Example 46: An electronic device of Example 45, wherein the obtained image is hyperspectral and/or plenoptic.

Example 47: An electronic device of Example 45, wherein the in-scene biological cells and/or substances include biological cells and/or substances that are characterized as: physically located on a surface; physically located in a medium; undisturbed in their environment; undisturbed and unadulterated; physically located on a surface in a manner that is undisturbed and unadulterated; not relocated for the purpose of image capture; unmanipulated for the purpose of image capture; on a surface that is unaffected by the scene-capture system.

Example 48: An electronic device of Example 45, wherein the biological cells are characterized as: cells of a multicell biological organism; cells of a tissue or organ of a multicell biological organism; cells of a tumor or growth multicell biological organism; single-celled organism; microbes; microscopic organisms; single-celled organism; living things that are too small to be seen with a human's naked eye; a biological creature that can only be seen by a human with mechanical magnification; microscopic spores; or a combination thereof.

Example 49: An electronic device of claim 45, wherein the biological cells are characterized as microbes that are characterized as: single-celled organisms; bacteria; archaea; fungi; mold; protists; viruses; microscopic multi-celled organisms; algae; bioagents; spores; germs; prions; a combination thereof.

Example 50: An electronic device of Example 45, wherein the pathobiological biological cells include: pathologic cells; diseased cells; cancer cells; infectious agents; pathogens; bioagents; disease-producing agents; combination thereof.

Example 51: An electronic device of Example 45, wherein the biological cells have a size range that is selected from a group consisting of: 10-100 nanometers (nm); 10-80 nm; 10-18 nm; 15-25 nm; and 50-150 nm.

Example 52: An electronic device of Example 45, wherein the scene includes: one or more surfaces on which the in-scene biological cells and/or substances inhabit; a liquid in which the in-scene biological cells and/or substances inhabit; a bodily fluid in which the in-scene biological cells and/or substances inhabit; an area in which the in-scene biological cells and/or substances inhabit; a volume in which the in-scene biological cells and/or substances inhabit; an area or volume with its dimensions falling below 0.1 mm; or a combination thereof.

Example 53: An electronic device of Example 45, wherein the obtained image is micrographic because the image of the scene is captured by the scene-capture system at least in part: on a microscopic scale; using a microscope like magnification; includes microscopic structures and features; includes structures and features that are not visible to a naked human eye; or a combination thereof.

Example 54: An electronic device of Example 45, wherein the obtained image is spectrographic at least in part because the scene-capture system captures the image of the scene using some portion of the electromagnetic spectrum as it interacts with matter, such interactions include, for example, absorption, emission, scattering, reflection, and refraction.

Example 55: An electronic device of Example 45, wherein the scene-capture system to obtain a sequence of images of the scene.

Example 56: An electronic device of Example 45, wherein the scene-capture system is characterized as: a camera; a digital camera; a still image camera; a video camera; a digital camera with micro-optics that optical magnification for obtaining the image; a digital camera with micro-optics are part of the electronic device itself; a digital camera with micro-optics are part of a smartdevice with its own processor and camera; a digital camera with micro-optics are part of additional camera functionality provided by an accessory or case for a smartdevice that operatively attaches to the smartdevice and adds the additional processing capabilities and functionalities for its scene-capture system; a flush mounted bead lens to perform optical magnification for its scene-capture system; a 12 or greater megapixel camera for its scene-capture system; a 12 or greater megapixel camera with flush mounted micro-optic bead lens to do optical magnification for its scene-capture system; a sapphire lens for its scene-capture system; captures an image using at least some portion of the visible electromagnetic spectrum; captures an image using at least some portion of the non-visible electromagnetic spectrum; captures an image using infrared light waves; captures an image using ultraviolet light waves; captures an image using ambient in-environment electromagnetic sources; captures images using ambient in-environment electromagnetic sources only; captures an image using artificial visible lighting which is added to the environment via artificial lighting sources associated with the scene-capture system; captures an image using artificial non-visible lighting which is added to the environment via artificial non-visible lighting sources associated with the scene-capture system; captures the image of the scene with optical magnification; captures the image of the scene with an optical magnification of a range of 20-200×; captures the image of the scene with an optical magnification of a range of 200-1000×; captures the image of the scene with an optical magnification of a range of 1000-5000×; captures the image of the scene with an optical magnification of a range of 2500-5000×; captures the image of the scene with an optical magnification of a range of 5000-10000×; captures the image of the scene with an optical magnification of greater than 1000×; captures the image of the scene with an optical magnification of greater than 5000×; captures a sequence of magnified images of the scene; enhances the image of the scene with digital magnification; enhances the image of the scene with digital image enhancement; enhances the image of the scene to a magnification level achievable by an electron scanning microscope (ESM); a combination thereof.

Example 57: An electronic device of Example 45 further comprising an environmental sensor system to obtain an environmental factor associated with the in-scene biological cells and/or substances or the environment surrounding the in-scene biological cells and/or substances.

Example 58: An electronic device of Example 57, wherein the obtained environmental factors includes biotic, abiotic, and associated factors.

Example 59: An electronic device of Example 57, wherein the environmental sensor system obtains the environmental factor by measurement or sensing.

Example 60: An electronic device of Example 57, wherein the report system to associate the obtained environmental factor with the obtained image and/or with the detected type or class of biological cell and/or substance.

Example 61: An electronic device of Example 57, wherein the obtained environmental factors are selected from a group consisting of: temperature; timestamp; humidity; barometric pressure; ambient sound; location; ambient electromagnetic activity; ambient lighting conditions; WiFi fingerprint; GPS location; airborne particle counter; chemical detection; gases; radiation; air quality; airborne particulate matter; atmospheric pressure; altitude; Geiger counter; proximity detection; magnetic sensor; rain gauge; seismometer; airflow; motion detection; ionization detection; gravity measurement; photoelectric sensor; piezo capacitive sensor; capacitance sensor; tilt sensor; angular momentum sensor; water-level detection; flame detector; smoke detector; force gauge; ambient electromagnetic sources; RFID detection; barcode reading; or a combination thereof.

Example 62: An electronic device of Example 45, wherein the detection of the biologic detection system includes operations to: access a database of signatures of pathobiological cells and/or substances; isolate a pathobiological cell and/or substance in the obtained image; correlate the isolated pathobiological cell and/or substance to at least one signature in the database; determine that the correlation is significant enough to indicate a sufficient degree of confidence to identify the isolated pathobiological cell and/or substance as being a pathobiological cell and/or substance; in response to that correlation determination, label the isolated pathobiological cell and/or substance as being the determined pathobiological cell and/or substance.

Example 63: An electronic device of Example 45, wherein the detection of the biologic detection system includes operations to: provide the obtained image to a trained pathobiological detection engine, the trained pathobiological detection engine being an AI/ML/DL engine trained to detect and/or identify pathobiological cells and/or substances based on a training corpus of signatures of pathobiological cells and/or substances; receive a positive indication from the pathobiological detection engine that the obtained image includes a pathobiological cell and/or substance therein and/or identity of that pathobiological cell and/or substance.

Example 64: An electronic device of Example 45, wherein the detection of the biologic detection system includes operations to: provide the obtained image to a trained pathobiological detection engine, the trained pathobiological detection engine being an AI/ML/DL engine trained to detect and/or identify pathobiological cells and/or substances based on a training corpus of signatures of pathobiological cells and/or substances; receive a positive indication from the pathobiological detection engine that the obtained image includes a pathobiological cell and/or substance therein and/or identity of that pathobiological cell and/or substance, wherein the obtained image includes data captured from the visible and/or non-visible electromagnetic spectrum of the scene.

Example 65: An electronic device of Example 45, wherein the report system to report an identification of the pathobiological cell and/or substance in the obtained image.

Example 66: An electronic device of Example 45, wherein the report of the report system is characterized by performing operations that: send a communication to a human or machine that is designated to receive such communications via wired or wireless communications mechanisms; send a notification to a human or machine that is designated to receive such notification via wired or wireless communications mechanisms; update a database designated to receive such updates via wired or wireless communications mechanisms; store the detection in a memory; a combination thereof.

Example 67: An electronic device of Example 45 further comprising an amelioration system to react to the detection and/or identification in a manner that ameliorates the neutralizes harmful effects of the detected and/or identified pathobiological cells and/or substances.

Example 68: An electronic device of Example 45 further comprising an amelioration system to react to the detection and/or identification in a manner that ameliorates the neutralizes harmful effects of the detected and/or identified pathobiological cells and/or substances, wherein the amelioration action is characterized as: introducing an active material to a physical location of the pathobiological cell and/or substance to neutralize the pathobiological nature of the detected/identified pathobiological cell and/or substance; dispatch or request a visit by a robot or human to a physical location of the pathobiological cell and/or substance to neutralize the pathobiological nature of the detected/identified pathobiological cell and/or substance; dispatch or request a visit by a robot or human to a physical location of the pathobiological cell and/or substance to document the conditions around the pathobiological cell and/or substance; activate an operation of a proximate electronic device or system that is proximate a physical location of the pathobiological cell and/or substance to the pathobiological nature of the detected/identified pathobiological cell and/or substance; activate an operation of a proximate camera that is proximate a physical location of the pathobiological cell and/or substance to document the area around the pathobiological cell and/or substance; a combination thereof.

Example 69: A method comprising: obtaining an image of a scene that includes in situ biological cells and/or substances therein, wherein the obtained image is micrographic, spectrographic, and/or digital; analyzing the obtained image and detecting pathobiological cells and/or substance amongst the in-scene biological cells and/or substances; reporting a detection the pathobiological cells and/or substances in the obtained image.

Example 70: A method of Example 69 further comprising: sensing an environmental factor associated with the in-scene biological cells and/or substances or the environment surrounding the in-scene biological cells and/or substances; associating the obtained environmental factor with the obtained image and/or with the detected pathobiological cell and/or substance.

Example 71: A method of Example 69, wherein the detection operation includes: providing the obtained image to a trained pathobiological detection engine, the trained pathobiological detection engine being an AI/ML/DL engine trained to detect and/or identify pathobiological cells and/or substances based on a training corpus of signatures of pathobiological cells and/or substances; receiving a positive indication from the pathobiological detection engine that the obtained image includes a pathobiological cell and/or substance therein and/or identity of that pathobiological cell and/or substance.

Example 72: A method of Example 69, wherein detection operation includes: providing the obtained image to a trained pathobiological detection engine, the trained pathobiological detection engine being an AI/ML/DL engine trained to detect and/or identify pathobiological cells and/or substances based on a training corpus of signatures of pathobiological cells and/or substances; receiving a positive indication from the pathobiological detection engine that the obtained image includes a pathobiological cell and/or substance therein and/or identity of that pathobiological cell and/or substance, wherein the obtained image includes data captured from the visible and/or non-visible electromagnetic spectrum of the scene.

Example 73: A non-transitory computer-readable storage medium comprising instructions that when executed cause a processor of a computing device to perform operations comprising: obtaining an image of a scene that includes in situ biological cells and/or substances therein, wherein the obtained image is micrographic, spectrographic, and/or digital; analyzing the obtained image and detecting pathobiological cells and/or substance amongst the in-scene biological cells and/or substances; reporting a detection the pathobiological cells and/or substances in the obtained image.

Example 74: Example X: A non-transitory computer-readable storage medium of Example 73, wherein the operations further comprise: sensing an environmental factor associated with the in-scene biological cells and/or substances or the environment surrounding the in-scene biological cells and/or substances; associating the obtained environmental factor with the obtained image and/or with the detected pathobiological cell and/or substance.

Example 75: Example X: A non-transitory computer-readable storage medium of Example 73, wherein the detection operation includes: providing the obtained image to a trained pathobiological detection engine, the trained pathobiological detection engine being an AI/ML/DL engine trained to detect and/or identify pathobiological cells and/or substances based on a training corpus of signatures of pathobiological cells and/or substances; receiving a positive indication from the pathobiological detection engine that the obtained image includes a pathobiological cell and/or substance therein and/or identity of that pathobiological cell and/or substance.

Example 76: A non-transitory computer-readable storage medium of Example 73, wherein the detection operation includes: providing the obtained image to a trained pathobiological detection engine, the trained pathobiological detection engine being an AI/ML/DL engine trained to detect and/or identify pathobiological cells and/or substances based on a training corpus of signatures of biological cells and/or substances; receiving a positive indication from the pathobiological detection engine that the obtained image includes a pathobiological cell and/or substance therein and/or identity of that pathobiological cell and/or substance, wherein the obtained image includes data captured from the visible and/or non-visible electromagnetic spectrum of the scene.

Example 77: An electronic device comprising: a scene-capture system to obtain an image of a scene that includes biological cells and/or substances therein; a biologic identification system to analyze the obtained image and identify one or more biological cells and/or substance amongst the in-scene biological cells and/or substances; a report system to report the identified the one or more biological cells and/or substances in the obtained image.

Example 78: An electronic device of Example 77, wherein the identified biological cells and/or substances is a member of a type or class of biological cells and/or substances that are pathobiological.

Example 79: An electronic device of Example 78, wherein the pathobiological biological cells includes: pathologic cells; diseased cells; cancer cells; infectious agents; pathogens; bioagents; disease-producing agents; or combination thereof.

Example 80: An electronic device of Example 77, wherein the in-scene biological cells and/or substances are in situ.

Example 81: An electronic device of Example 77, wherein the obtained image is micrographic, spectrographic, and/or digital.

Example 82: An electronic device of Example 81, wherein the obtained image is micrographic because the image of the scene is captured by the scene-capture system at least in part: on a microscopic scale; using a microscope like magnification; includes microscopic structures and features; includes structures and features that are not visible to a naked human eye; or a combination thereof.

Example 83: An electronic device of Example 81, wherein the obtained image is spectrographic at least in part because the scene-capture system captures the image of the scene using some portion of the electromagnetic spectrum as it interacts with matter, such interactions include, for example, absorption, emission, scattering, reflection, and refraction.

Example 84: An electronic device of Example 77, wherein the obtained image is hyperspectral and/or plenoptic.

Example 85: An electronic device of Example 77, wherein the in-scene biological cells and/or substances include biological cells and/or substances that are characterized as: physically located on a surface; physically located in a medium; undisturbed in their environment; undisturbed and unadulterated; physically located on a surface in a manner that is undisturbed and unadulterated; not relocated for the purpose of image capture; unmanipulated for the purpose of image capture; or on a surface that is unaffected by the scene-capture system.

Example 86: An electronic device of Example 77, wherein the biological cells are characterized as: cells of a multicell biological organism; cells of a tissue or organ of a multicell biological organism; cells of a tumor or growth multicell biological organism; single-celled organism; microbes; microscopic organisms; single-celled organism; living things that are too small to be seen with a human's naked eye; a biological creature that can only be seen by a human with mechanical magnification; microscopic spores; or a combination thereof.

Example 87: An electronic device of Example 77, wherein the biological cells are characterized as microbes that are characterized as: single-celled organisms; bacteria; archaea; fungi; mold; protists; viruses; microscopic multi-celled organisms; algae; bioagents; spores; germs; prions; or a combination thereof.

Example 88: An electronic device of Example 77, wherein the biological cells have a size range that is selected from a group consisting of: 10-100 nanometers (nm); 10-80 nm; 10-18 nm; 15-25 nm; and 50-150 nm.

Example 89: An electronic device of Example 77, wherein the scene includes: one or more surfaces on which the in-scene biological cells and/or substances inhabit; a liquid in which the in-scene biological cells and/or substances inhabit; a bodily fluid in which the in-scene biological cells and/or substances inhabit; an area in which the in-scene biological cells and/or substances inhabit; a volume in which the in-scene biological cells and/or substances inhabit; an area or volume with its dimensions falling below 0.1 mm; or a combination thereof.

Example 90: An electronic device of Example 77, wherein the scene-capture system to obtain a sequence of images of the scene.

Example 91: An electronic device of Example 77, wherein the scene-capture system is characterized as: a camera; a digital camera; a still image camera; a video camera; a digital camera with micro-optics that optical magnification for obtaining the image; a digital camera with micro-optics are part of the electronic device itself; a digital camera with micro-optics are part of a smartdevice with its own processor and camera; a digital camera with micro-optics are part of additional camera functionality provided by an accessory or case for a smartdevice that operatively attaches to the smartdevice and adds the additional processing capabilities and functionalities for its scene-capture system; a flush mounted bead lens to perform optical magnification for its scene-capture system; a 12 or greater megapixel camera for its scene-capture system; a 12 or greater megapixel camera with flush mounted micro-optic bead lens to do optical magnification for its scene-capture system; a sapphire lens for its scene-capture system; captures an image using at least some portion of the visible electromagnetic spectrum; captures an image using at least some portion of the non-visible electromagnetic spectrum; captures an image using infrared light waves; captures an image using ultraviolet light waves; captures an image using ambient in-environment electromagnetic sources; captures images using ambient in-environment electromagnetic sources only; captures an image using artificial visible lighting which is added to the environment via artificial lighting sources associated with the scene-capture system; captures an image using artificial non-visible lighting which is added to the environment via artificial non-visible lighting sources associated with the scene-capture system; captures the image of the scene with optical magnification; captures the image of the scene with an optical magnification of a range of 20-200×; captures the image of the scene with an optical magnification of a range of 200-1000×; captures the image of the scene with an optical magnification of a range of 1000-5000×; captures the image of the scene with an optical magnification of a range of 2500-5000×; captures the image of the scene with an optical magnification of a range of 5000-10000×; captures the image of the scene with an optical magnification of greater than 1000×; captures the image of the scene with an optical magnification of greater than 5000×; captures a sequence of magnified images of the scene; enhances the image of the scene with digital magnification; enhances the image of the scene with digital image enhancement; enhances the image of the scene to a magnification level achievable by an electron scanning microscope (ESM); a combination thereof.

Example 92: An electronic device of Example 77 further comprising an environmental sensor system to obtain an environmental factor associated with the in-scene biological cells and/or substances or the environment surrounding the in-scene biological cells and/or substances.

Example 93: An electronic device of Example 92, wherein the obtained environmental factors includes biotic, abiotic, and associated factors.

Example 94: An electronic device of Example 92, wherein the environmental sensor system obtains the environmental factor by measurement or sensing.

Example 95: An electronic device of Example 92, wherein the report system to associate the obtained environmental factor with the obtained image and/or with the detected type or class of biological cell and/or substance.

Example 96: An electronic device of Example 92, wherein the obtained environmental factors are selected from a group consisting of: temperature; timestamp; humidity; barometric pressure; ambient sound; location; ambient electromagnetic activity; ambient lighting conditions; WiFi fingerprint; GPS location; airborne particle counter; chemical detection; gases; radiation; air quality; airborne particulate matter; atmospheric pressure; altitude; Geiger counter; proximity detection; magnetic sensor; rain gauge; seismometer; airflow; motion detection; ionization detection; gravity measurement; photoelectric sensor; piezo capacitive sensor; capacitance sensor; tilt sensor; angular momentum sensor; water-level detection; flame detector; smoke detector; force gauge; ambient electromagnetic sources; RFID detection; barcode reading; or a combination thereof.

Example 97: An electronic device of Example 77, wherein the identification of the biologic system includes operations to: access a database of signatures of biological cells and/or substances; isolate a biological cell and/or substance in the obtained image; correlate the isolated biological cell and/or substance to at least one signature in the database; determine that the correlation is significant enough to indicate a sufficient degree of confidence to identify the isolated biological cell and/or substance as being a biological cell and/or substance; in response to that correlation determination, label the isolated biological cell and/or substance as being the determined biological cell and/or sub stance.

Example 98: An electronic device of Example 77, wherein the identification of the biologic detection system includes operations to: provide the obtained image to a trained biological identification engine, the trained biological identification engine being an AI/ML/DL engine trained to detect and/or identify biological cells and/or substances based on a training corpus of signatures of biological cells and/or substances; receive a positive indication from the biological identification engine that the obtained image includes a biological cell and/or substance therein and/or identity of that biological cell and/or substance.

Example 99: An electronic device of Example 77, wherein the identification of the biologic detection system includes operations to: provide the obtained image to a trained biological identification engine, the trained biological identification engine being an AI/ML/DL engine trained to detect and/or identify biological cells and/or substances based on a training corpus of signatures of biological cells and/or substances; receive a positive indication from the biological identification engine that the obtained image includes a biological cell and/or substance therein and/or identity of that biological cell and/or substance, wherein the obtained image includes data captured from the visible and/or non-visible electromagnetic spectrum of the scene.

Example 100: An electronic device of Example 77, wherein the report system to report an identification of the biological cell and/or substance in the obtained image.

Example 101: An electronic device of Example 77, wherein the report of the report system is characterized by performing operations that: send a communication to a human or machine that is designated to receive such communications via wired or wireless communications mechanisms; send a notification to a human or machine that is designated to receive such notification via wired or wireless communications mechanisms; update a database designated to receive such updates via wired or wireless communications mechanisms; store the detection in a memory; a combination thereof.

Example 102: An electronic device of Example 77, wherein the report of the report system indicates that the type of biological cells and/or substances in the obtained image is a category flagged for further research and inquiry.

Example 103: An electronic device of Example 77 further comprising an amelioration system to react to the detection and/or identification in a manner that ameliorates the neutralizes harmful effects of the detected and/or identified biological cells and/or substances.

Example 104: An electronic device of Example 77 further comprising an amelioration system to react to the detection and/or identification in a manner that ameliorates the neutralizes harmful effects of the detected and/or identified biological cells and/or substances, wherein the amelioration action is characterized as: introducing an active material to a physical location of the biological cell and/or substance to neutralize the biological nature of the detected/identified biological cell and/or substance; dispatch or request a visit by a robot or human to a physical location of the biological cell and/or substance to neutralize the biological nature of the detected/identified biological cell and/or substance; dispatch or request a visit by a robot or human to a physical location of the biological cell and/or substance to document the conditions around the biological cell and/or substance; activate an operation of a proximate electronic device or system that is proximate a physical location of the biological cell and/or substance to neutralize the biological nature of the detected/identified biological cell and/or substance; activate an operation of a proximate camera that is proximate a physical location of the biological cell and/or substance to document the area around the biological cell and/or substance; a combination thereof.

Example 105: A method comprising: obtaining an image of a scene that includes biological cells and/or substances therein, wherein the obtained image is micrographic, spectrographic, and/or digital; analyzing the obtained image and identifying one or more biological cells and/or substance amongst the in-scene biological cells and/or substances; reporting the one or more identified biological cells and/or substances in the obtained image.

Example 106: A method of Example 105, wherein the one or more identified biological cells and/or substances is a pathobiological.

Example 107: A method of Example 105, wherein the in-scene biological cells and/or substances are in situ.

Example 108: A method of Example 105 further comprising: sensing an environmental factor associated with the in-scene biological cells and/or substances or the environment surrounding the in-scene biological cells and/or substances; associating the obtained environmental factor with the obtained image and/or with the one or more identified biological cells and/or substances.

Example 109: A method of Example 105, wherein the detection operation includes: providing the obtained image to a trained biological detection engine, the trained biological identification engine being an AI/ML/DL engine trained to detect and/or identify biological cells and/or substances based on a training corpus of signatures of biological cells and/or substances; receiving a positive indication from the biological identification engine that the obtained image includes a biological cell and/or substance therein and/or identity of that biological cell and/or substance.

Example 110: A method of Example 105, wherein detection operation includes: providing the obtained image to a trained biological identification engine, the trained biological identification engine being an AI/ML/DL engine trained to detect and/or identify biological cells and/or substances based on a training corpus of signatures of biological cells and/or substances; receiving a positive indication from the biological identification engine that the obtained image includes a biological cell and/or substance therein and/or identity of that biological cell and/or substance, wherein the obtained image includes data captured from the visible and/or non-visible electromagnetic spectrum of the scene.

Example 111: A non-transitory computer-readable storage medium comprising instructions that when executed cause a processor of a computing device to perform operations comprising: obtaining an image of a scene that includes biological cells and/or substances therein, wherein the obtained image is micrographic, spectrographic, and/or digital; analyzing the obtained image and identifying one or more biological cells and/or substance amongst the in-scene biological cells and/or substances; reporting the one or more identified biological cells and/or substances in the obtained image.

Example 112: A non-transitory computer-readable storage medium of Example 111, wherein the one or more identified biological cells and/or substances is a pathobiological.

Example 113: A non-transitory computer-readable storage medium of Example 111, wherein the in-scene biological cells and/or substances are in situ.

Example 114: A non-transitory computer-readable storage medium of Example 111, wherein the operations further comprise: sensing an environmental factor associated with the in-scene biological cells and/or substances or the environment surrounding the in-scene biological cells and/or substances; associating the obtained environmental factor with the obtained image and/or with the one or more identified biological cells and/or substances.

Example 115: A non-transitory computer-readable storage medium of Example 111, wherein the detection operation includes: providing the obtained image to a trained biological identification engine, the trained biological identification engine being an AI/ML/DL engine trained to detect and/or identify biological cells and/or substances based on a training corpus of signatures of biological cells and/or substances; receiving a positive indication from the biological identification engine that the obtained image includes a biological cell and/or substance therein and/or identity of that biological cell and/or substance.

Example 116: A non-transitory computer-readable storage medium of Example 111, wherein the detection operation includes: providing the obtained image to a trained biological identification engine, the trained biological identification engine being an AI/ML/DL engine trained to detect and/or identify biological cells and/or substances based on a training corpus of signatures of biological cells and/or substances; receiving a positive indication from the biological identification engine that the obtained image includes a biological cell and/or substance therein and/or identity of that biological cell and/or substance, wherein the obtained image includes data captured from the visible and/or non-visible electromagnetic spectrum of the scene.

What is claimed is:

1. An electronic device comprising:
a scene-capture system to obtain an image of a scene that includes biological cells and/or substances therein;
a biologic detection system to analyze the obtained image and detect a type or class of biological cells and/or substance amongst the in-scene biological cells and/or substances;
a report system to report detection of the type of biological cells and/or substances in the obtained image.

2. An electronic device of claim 1, wherein the type or class of detected biological cell and/or substance is a pathobiological cell and/or substance.

3. An electronic device of claim 2, wherein the pathobiological biological cells includes:
pathologic cells;
diseased cells;
cancer cells;
infectious agents;
pathogens;
bioagents;
disease-producing agents;
combination thereof.

4. An electronic device of claim 1, wherein the in-scene biological cells and/or substances are in situ.

5. An electronic device of claim 1, wherein the obtained image is micrographic, spectrographic, and/or digital.

6. An electronic device of claim 5, wherein the obtained image is micrographic because the image of the scene is captured by the scene-capture system at least in part:
on a microscopic scale;
using a microscope like magnification;
includes microscopic structures and features; or
includes structures and features that are not visible to a naked human eye; or a combination thereof.

7. An electronic device of claim 5, wherein the obtained image is spectrographic at least in part because the scene-capture system captures the image of the scene using some portion of the electromagnetic spectrum as it interacts with matter, such interactions include absorption, emission, scattering, reflection, and/or refraction.

8. An electronic device of claim 1, wherein the obtained image is hyperspectral and/or plenoptic.

9. An electronic device of claim 1, wherein the scene-capture system employs a near-field scanning optical microscope to obtain the image.

10. An electronic device of claim 1, wherein the in-scene biological cells and/or substances include biological cells and/or substances that are characterized as:
physically located on a surface;
physically located in a medium;
undisturbed in their environment;
undisturbed and unadulterated;
physically located on a surface in a manner that is undisturbed and unadulterated;
not relocated for the purpose of image capture;
unmanipulated for the purpose of image capture; or
on a surface that is unaffected by the scene-capture system.

11. An electronic device of claim 1, wherein the biological cells are characterized as:
cells of a multicell biological organism;
cells of a tissue or organ of a multicell biological organism;
cells of a tumor or growth multicell biological organism;
single-celled organism;
microbes;
microscopic organisms;
single-celled organism;
living things that are too small to be seen with a human's naked eye;
a biological creature that can only be seen by a human with mechanical magnification;
microscopic spores; or
a combination thereof.

12. An electronic device of claim 1, wherein the biological cells are characterized as microbes that are characterized as:
single-celled organisms;
bacteria;
archaea;
fungi;
mold;
protists;
viruses;
microscopic multi-celled organisms;
algae;
bioagents;
spores;
germs;
prions; or
a combination thereof.

13. An electronic device of claim 1, wherein the biological cells have a size range that is selected from a group consisting of:
10-100 nanometers (nm);
10-80 nm;
10-18 nm;
15-25 nm; and
50-150 nm.

14. An electronic device of claim 1, wherein the scene includes:
one or more surfaces on which the in-scene biological cells and/or substances inhabit;
a liquid in which the in-scene biological cells and/or substances inhabit;
a bodily fluid in which the in-scene biological cells and/or substances inhabit;
an area in which the in-scene biological cells and/or substances inhabit;
a volume in which the in-scene biological cells and/or substances inhabit;
an area or volume with its dimensions falling below 0.1 mm; or
a combination thereof.

15. An electronic device of claim 1, wherein the scene-capture system to obtain a sequence of images of the scene.

16. An electronic device of claim 1, wherein the scene-capture system is characterized as:
a camera;
a digital camera;
a still image camera;
a video camera;
a digital camera with micro-optics that optical magnification for obtaining the image;
a digital camera with micro-optics are part of the electronic device itself;
a digital camera with micro-optics are part of a smartdevice with its own processor and camera;
a digital camera with micro-optics are part of additional camera functionality provided by an accessory or case for a smartdevice that operatively attaches to the smartdevice and adds the additional processing capabilities and functionalities for its scene-capture system;
a flush mounted bead lens to perform optical magnification for its scene-capture system;
a 12 or greater megapixel camera for its scene-capture system;
a 12 or greater megapixel camera with flush mounted micro-optic bead lens to do optical magnification for its scene-capture system;
a sapphire lens for its scene-capture system;
captures an image using at least some portion of the visible electromagnetic spectrum;
captures an image using at least some portion of the non-visible electromagnetic spectrum;
captures an image using infrared light waves;
captures an image using ultraviolet light waves;
captures an image using ambient in-environment electromagnetic sources;
captures images using ambient in-environment electromagnetic sources only;
captures an image using artificial visible lighting which is added to the environment via artificial lighting sources associated with the scene-capture system;
captures an image using artificial non-visible lighting which is added to the environment via artificial non-visible lighting sources associated with the scene-capture system;
captures the image of the scene with optical magnification;
captures the image of the scene with an optical magnification of a range of 20-200×;
captures the image of the scene with an optical magnification of a range of 200-1000×;
captures the image of the scene with an optical magnification of a range of 1000-5000×;
captures the image of the scene with an optical magnification of a range of 2500-5000×;
captures the image of the scene with an optical magnification of a range of 5000-10000×;
captures the image of the scene with an optical magnification of greater than 1000×;
captures the image of the scene with an optical magnification of greater than 5000×;
captures a sequence of magnified images of the scene;
enhances the image of the scene with digital magnification;
enhances the image of the scene with digital image enhancement;
enhances the image of the scene to a magnification level achievable by an electron scanning microscope (ESM); or
a combination thereof.

17. An electronic device of claim 1, further comprising an environmental sensor system to obtain an environmental factor associated with the in-scene biological cells and/or substances or the environment surrounding the in-scene biological cells and/or substances.

18. An electronic device of claim 17, wherein the obtained environmental factors includes biotic, abiotic, and associated factors.

19. An electronic device of claim 17, wherein the environmental sensor system obtains the environmental factor by measurement or sensing.

20. An electronic device of claim 17, wherein the report system to associate the obtained environmental factor with the obtained image and/or with the detected type or class of biological cell and/or substance.

21. An electronic device of claim 17, wherein the obtained environmental factors are selected from a group consisting of:
temperature;
timestamp;
humidity;
barometric pressure;
ambient sound;
location;
ambient electromagnetic activity;
ambient lighting conditions;
WiFi fingerprint;
GPS location;
airborne particle counter;
chemical detection;
gases;
radiation;
air quality;
airborne particulate matter;
atmospheric pressure;
altitude;
Geiger counter;
proximity detection;
magnetic sensor;
rain gauge;
seismometer;
airflow;
motion detection;
ionization detection;
gravity measurement;
photoelectric sensor;
piezo capacitive sensor;
capacitance sensor;
tilt sensor;
angular momentum sensor;
water-level detection;
flame detector;
smoke detector;
force gauge;
ambient electromagnetic sources;
RFID detection;
barcode reading; and
a combination thereof.

22. An electronic device of claim 1, wherein the detection of the biologic system includes operations to:
access a database of signatures of biological cells and/or substances;
isolate a biological cell and/or substance in the obtained image;
correlate the isolated biological cell and/or substance to at least one signature in the database;
determine that the correlation is significant enough to indicate a sufficient degree of confidence to identify the isolated biological cell and/or substance as being a biological cell and/or substance;
in response to that correlation determination, label the isolated biological cell and/or substance as being the determined biological cell and/or substance.

23. An electronic device of claim 1, wherein the detection of the biologic detection system includes operations to:
provide the obtained image to a trained biological detection engine, the trained biological detection engine being an AI/ML/DL engine trained to detect and/or identify biological cells and/or substances based on a training corpus of signatures of biological cells and/or substances;
receive a positive indication from the biological detection engine that the obtained image includes a biological cell and/or substance therein and/or identity of that biological cell and/or substance.

24. An electronic device of claim 1, wherein the detection of the biologic detection system includes operations to:
provide the obtained image to a trained biological detection engine, the trained biological detection engine being an AI/ML/DL engine trained to detect and/or identify biological cells and/or substances based on a training corpus of signatures of biological cells and/or substances;
receive a positive indication from the biological detection engine that the obtained image includes a biological cell and/or substance therein and/or identity of that biological cell and/or substance,
wherein the obtained image includes data captured from the visible and/or non-visible electromagnetic spectrum of the scene.

25. An electronic device of claim 1, wherein the report system to report an identification of the biological cell and/or substance in the obtained image.

26. An electronic device of claim 1, wherein the report of the report system is characterized by performing operations that:
send a communication to a human or machine that is designated to receive such communications via wired or wireless communications mechanisms;
send a notification to a human or machine that is designated to receive such notification via wired or wireless communications mechanisms;
update a database designated to receive such updates via wired or wireless communications mechanisms;
store the detection in a memory; or
a combination thereof.

27. An electronic device of claim 1, wherein the report of the report system indicates that the type of biological cells and/or substances in the obtained image is a category flagged for further research and inquiry.

28. An electronic device of claim 1, further comprising an amelioration system to react to the detection and/or identification in a manner that ameliorates the neutralizes harmful effects of the detected and/or identified biological cells and/or substances.

29. An electronic device of claim 1, further comprising an amelioration system to react to the detection and/or identification in a manner that ameliorates the neutralizes harmful effects of the detected and/or identified biological cells and/or substances, wherein the amelioration action is characterized as:
introducing an active material to a physical location of the biological cell and/or substance to neutralize the biological nature of the detected/identified biological cell and/or substance;
dispatch or request a visit by a robot or human to a physical location of the biological cell and/or substance to neutralize the biological nature of the detected/identified biological cell and/or substance;
dispatch or request a visit by a robot or human to a physical location of the biological cell and/or substance to document the conditions around the biological cell and/or substance;
activate an operation of a proximate electronic device or system that is proximate a physical location of the biological cell and/or substance to neutralize the biological nature of the detected/identified biological cell and/or substance;
activate an operation of a proximate camera that is proximate a physical location of the biological cell and/or substance to document the area around the biological cell and/or substance; or
a combination thereof.

30. An electronic device of claim 1, wherein, in whole or in part, the electronic device is selected from a group consisting of: a system-on-a-chip; a device-on-a-chip; a smartdevice; a computer; an ambulatory device; a microscope; a mobile device; and a wireless device.

31. An electronic device of claim 1, wherein the biologic detection system transmits the obtained image or a portion thereof across a communications network to a remote computer system.

32. An electronic device of claim 1, wherein the scene includes photonic nanostructures therein.

33. A method comprising:
obtaining an image of a scene that includes biological cells and/or substances therein, wherein the obtained image is micrographic, spectrographic, and/or digital;
analyzing the obtained image and detecting a type or class of biological cells and/or substance amongst the in-scene biological cells and/or substances;
reporting detection of the type of biological cells and/or substances in the obtained image.

34. A method of claim 33, wherein the type or class of detected biological cell and/or substance is a pathobiological cell and/or substance.

35. A method of claim 33, wherein the in-scene biological cells and/or substances are in situ.

36. A method of claim 33, further comprising:
sensing an environmental factor associated with the in-scene biological cells and/or substances or the environment surrounding the in-scene biological cells and/or substances;
associating the obtained environmental factor with the obtained image and/or with the detected type or class of biological cell and/or substance.

37. A method of claim 33, wherein the detection operation includes:
providing the obtained image to a trained biological detection engine, the trained biological detection engine being an AI/ML/DL engine trained to detect and/or identify biological cells and/or substances based on a training corpus of signatures of biological cells and/or substances;
receiving a positive indication from the biological detection engine that the obtained image includes a biological cell and/or substance therein and/or identity of that biological cell and/or substance.

38. A method of claim 33, wherein detection operation includes:
providing the obtained image to a trained biological detection engine, the trained biological detection engine being an AI/ML/DL engine trained to detect and/or identify biological cells and/or substances based on a training corpus of signatures of biological cells and/or substances;
receiving a positive indication from the biological detection engine that the obtained image includes a biological cell and/or substance therein and/or identity of that biological cell and/or substance,
wherein the obtained image includes data captured from the visible and/or non-visible electromagnetic spectrum of the scene.

39. A non-transitory computer-readable storage medium comprising instructions that when executed cause a processor of a computing device to perform operations comprising:
obtaining an image of a scene that includes biological cells and/or substances therein, wherein the obtained image is micrographic, spectrographic, and/or digital;
analyzing the obtained image and detecting a type or class of biological cells and/or substance amongst the in-scene biological cells and/or substances;
reporting detection of the type of biological cells and/or substances in the obtained image.

40. A non-transitory computer-readable storage medium of claim 39, wherein the type or class of detected biological cell and/or substance is a pathobiological cell and/or substance.

41. A non-transitory computer-readable storage medium of claim 39, wherein the in-scene biological cells and/or substances are in situ.

42. A non-transitory computer-readable storage medium of claim 39, wherein the operations further comprise:
sensing an environmental factor associated with the in-scene biological cells and/or substances or the environment surrounding the in-scene biological cells and/or substances;
associating the obtained environmental factor with the obtained image and/or with the detected type or class of biological cell and/or substance.

43. A non-transitory computer-readable storage medium of claim 39, wherein the detection operation includes:
providing the obtained image to a trained biological detection engine, the trained biological detection engine being an AI/ML/DL engine trained to detect and/or identify biological cells and/or substances based on a training corpus of signatures of biological cells and/or substances;
receiving a positive indication from the biological detection engine that the obtained image includes a biological cell and/or substance therein and/or identity of that biological cell and/or substance.

44. A non-transitory computer-readable storage medium of claim 39, wherein the detection operation includes:
providing the obtained image to a trained biological detection engine, the trained biological detection engine being an AI/ML/DL engine trained to detect and/or identify biological cells and/or substances based on a training corpus of signatures of biological cells and/or substances;
receiving a positive indication from the biological detection engine that the obtained image includes a biological cell and/or substance therein and/or identity of that biological cell and/or substance,
wherein the obtained image includes data captured from the visible and/or non-visible electromagnetic spectrum of the scene.

45. An electronic device comprising:
a scene-capture system to obtain an image of a scene that includes in situ biological cells and/or substances therein, wherein the obtained image is micrographic, spectrographic, and/or digital;
a biologic detection system to analyze the obtained image and detect pathobiological cells and/or substance amongst the in-scene biological cells and/or substances;
a report system to report a detection of the pathobiological cells and/or substances in the obtained image.

46. An electronic device of claim 45, wherein the obtained image is hyperspectral and/or plenoptic.

47. An electronic device of claim 45, wherein the in-scene biological cells and/or substances include biological cells and/or substances that are characterized as:
physically located on a surface;
physically located in a medium;
undisturbed in their environment;
undisturbed and unadulterated;
physically located on a surface in a manner that is undisturbed and unadulterated;
not relocated for the purpose of image capture;
unmanipulated for the purpose of image capture;
on a surface that is unaffected by the scene-capture system.

48. An electronic device of claim 45, wherein the biological cells are characterized as:
cells of a multicell biological organism;
cells of a tissue or organ of a multicell biological organism;
cells of a tumor or growth multicell biological organism;
single-celled organism;
microbes;
microscopic organisms;
single-celled organism;
living things that are too small to be seen with a human's naked eye;
a biological creature that can only be seen by a human with mechanical magnification;
microscopic spores; or
a combination thereof.

49. An electronic device of claim 45, wherein the biological cells are characterized as microbes that are characterized as:
single-celled organisms;
bacteria;
archaea;
fungi;
mold;
protists;
viruses;
microscopic multi-celled organisms;
algae;
bioagents;
spores;
germs;
prions; or
a combination thereof.

50. An electronic device of claim 45, wherein the pathobiological biological cells includes:
pathologic cells;
diseased cells;
cancer cells;
infectious agents;
pathogens;
bioagents;
disease-producing agents; or
combination thereof.

51. An electronic device of claim 45, wherein the biological cells have a size range that is selected from a group consisting of:
10-100 nanometers (nm);
10-80 nm;
10-18 nm;
15-25 nm; and
50-150 nm.

52. An electronic device of claim 45, wherein the scene includes:
one or more surfaces on which the in-scene biological cells and/or substances inhabit;
a liquid in which the in-scene biological cells and/or substances inhabit;
a bodily fluid in which the in-scene biological cells and/or substances inhabit;
an area in which the in-scene biological cells and/or substances inhabit;
a volume in which the in-scene biological cells and/or substances inhabit;
an area or volume with its dimensions falling below 0.1 mm; or
a combination thereof.

53. An electronic device of claim 45, wherein the obtained image is micrographic because the image of the scene is captured by the scene-capture system at least in part:
on a microscopic scale;
using a microscope like magnification;
includes microscopic structures and features;
includes structures and features that are not visible to a naked human eye; or
a combination thereof.

54. An electronic device of claim 45, wherein the obtained image is spectrographic at least in part because the scene-capture system captures the image of the scene using some portion of the electromagnetic spectrum as it interacts with matter, such interactions include absorption, emission, scattering, reflection, and/or refraction.

55. An electronic device of claim 45, wherein the scene-capture system to obtain a sequence of images of the scene.

56. An electronic device of claim 45, wherein the scene-capture system is characterized as:
a camera;
a digital camera;
a still image camera;
a video camera;
a digital camera with micro-optics that optical magnification for obtaining the image;
a digital camera with micro-optics are part of the electronic device itself;
a digital camera with micro-optics are part of a smartdevice with its own processor and camera;
a digital camera with micro-optics are part of additional camera functionality provided by an accessory or case for a smartdevice that operatively attaches to the smartdevice and adds the additional processing capabilities and functionalities for its scene-capture system;
a flush mounted bead lens to perform optical magnification for its scene-capture system;
a 12 or greater megapixel camera for its scene-capture system;
a 12 or greater megapixel camera with flush mounted micro-optic bead lens to do optical magnification for its scene-capture system;
a sapphire lens for its scene-capture system;
captures an image using at least some portion of the visible electromagnetic spectrum;
captures an image using at least some portion of the non-visible electromagnetic spectrum;
captures an image using infrared light waves;
captures an image using ultraviolet light waves;
captures an image using ambient in-environment electromagnetic sources;
captures images using ambient in-environment electromagnetic sources only;
captures an image using artificial visible lighting which is added to the environment via artificial lighting sources associated with the scene-capture system;
captures an image using artificial non-visible lighting which is added to the environment via artificial non-visible lighting sources associated with the scene-capture system;
captures the image of the scene with optical magnification;
captures the image of the scene with an optical magnification of a range of 20-200×;
captures the image of the scene with an optical magnification of a range of 200-1000×;
captures the image of the scene with an optical magnification of a range of 1000-5000×;
captures the image of the scene with an optical magnification of a range of 2500-5000×;
captures the image of the scene with an optical magnification of a range of 5000-10000×;
captures the image of the scene with an optical magnification of greater than 1000×;
captures the image of the scene with an optical magnification of greater than 5000×;
captures a sequence of magnified images of the scene;
enhances the image of the scene with digital magnification;
enhances the image of the scene with digital image enhancement;
enhances the image of the scene to a magnification level achievable by an electron scanning microscope (ESM); or
a combination thereof.

57. An electronic device of claim 45, further comprising an environmental sensor system to obtain an environmental factor associated with the in-scene biological cells and/or substances or the environment surrounding the in-scene biological cells and/or substances.

58. An electronic device of claim 57, wherein the obtained environmental factors includes biotic, abiotic, and associated factors.

59. An electronic device of claim 57, wherein the environmental sensor system obtains the environmental factor by measurement or sensing.

60. An electronic device of claim 57, wherein the report system to associate the obtained environmental factor with the obtained image and/or with the detected type or class of biological cell and/or substance.

61. An electronic device of claim 57, wherein the obtained environmental factors are selected from a group consisting of:
temperature;
timestamp;
humidity;
barometric pressure;
ambient sound;
location;
ambient electromagnetic activity;
ambient lighting conditions;
WiFi fingerprint;
GPS location;
airborne particle counter;
chemical detection;
gases;
radiation;
air quality;
airborne particulate matter;
atmospheric pressure;
altitude;
Geiger counter;
proximity detection;
magnetic sensor;
rain gauge;
seismometer;
airflow;
motion detection;
ionization detection;
gravity measurement;
photoelectric sensor;
piezo capacitive sensor;
capacitance sensor;
tilt sensor;
angular momentum sensor;
water-level detection;
flame detector;
smoke detector;
force gauge;
ambient electromagnetic sources;
RFID detection;
barcode reading; and
a combination thereof.

62. An electronic device of claim 45, wherein the detection of the biologic detection system includes operations to:
access a database of signatures of pathobiological cells and/or substances;
isolate a pathobiological cell and/or substance in the obtained image;
correlate the isolated pathobiological cell and/or substance to at least one signature in the database;
determine that the correlation is significant enough to indicate a sufficient degree of confidence to identify the isolated pathobiological cell and/or substance as being a pathobiological cell and/or substance;
in response to that correlation determination, label the isolated pathobiological cell and/or substance as being the determined pathobiological cell and/or substance.

63. An electronic device of claim 45, wherein the detection of the biologic detection system includes operations to:
provide the obtained image to a trained pathobiological detection engine, the trained pathobiological detection engine being an AI/ML/DL engine trained to detect and/or identify pathobiological cells and/or substances based on a training corpus of signatures of pathobiological cells and/or substances;
receive a positive indication from the pathobiological detection engine that the obtained image includes a pathobiological cell and/or substance therein and/or identity of that pathobiological cell and/or substance.

64. An electronic device of claim 45, wherein the detection of the biologic detection system includes operations to:
provide the obtained image to a trained pathobiological detection engine, the trained pathobiological detection engine being an AI/ML/DL engine trained to detect and/or identify pathobiological cells and/or substances based on a training corpus of signatures of pathobiological cells and/or substances;
receive a positive indication from the pathobiological detection engine that the obtained image includes a pathobiological cell and/or substance therein and/or identity of that pathobiological cell and/or substance,
wherein the obtained image includes data captured from the visible and/or non-visible electromagnetic spectrum of the scene.

65. An electronic device of claim 45, wherein the report system to report an identification of the pathobiological cell and/or substance in the obtained image.

66. An electronic device of claim 45, wherein the report of the report system is characterized by performing operations that:
send a communication to a human or machine that is designated to receive such communications via wired or wireless communications mechanisms;
send a notification to a human or machine that is designated to receive such notification via wired or wireless communications mechanisms;
update a database designated to receive such updates via wired or wireless communications mechanisms;
store the detection in a memory; or
a combination thereof.

67. An electronic device of claim 45, further comprising an amelioration system to react to the detection and/or identification in a manner that ameliorates the neutralizes harmful effects of the detected and/or identified pathobiological cells and/or substances.

68. An electronic device of claim 45, further comprising an amelioration system to react to the detection and/or identification in a manner that ameliorates the neutralizes harmful effects of the detected and/or identified pathobiological cells and/or substances, wherein the amelioration action is characterized as:
introducing an active material to a physical location of the pathobiological cell and/or substance to neutralize the pathobiological nature of the detected/identified pathobiological cell and/or substance;
dispatch or request a visit by a robot or human to a physical location of the pathobiological cell and/or substance to neutralize the pathobiological nature of the detected/identified pathobiological cell and/or substance;
dispatch or request a visit by a robot or human to a physical location of the pathobiological cell and/or substance to document the conditions around the pathobiological cell and/or substance;
activate an operation of a proximate electronic device or system that is proximate a physical location of the pathobiological cell and/or substance to the pathobiological nature of the detected/identified pathobiological cell and/or substance;
activate an operation of a proximate camera that is proximate a physical location of the pathobiological cell and/or substance to document the area around the pathobiological cell and/or substance; or
a combination thereof.

69. A method comprising:
obtaining an image of a scene that includes in situ biological cells and/or substances therein, wherein the obtained image is micrographic, spectrographic, and/or digital;
analyzing the obtained image and detecting pathobiological cells and/or substance amongst the in-scene biological cells and/or substances;
reporting a detection the pathobiological cells and/or substances in the obtained image.

70. A method of claim 69 further comprising:
sensing an environmental factor associated with the in-scene biological cells and/or substances or the environment surrounding the in-scene biological cells and/or substances;
associating the obtained environmental factor with the obtained image and/or with the detected pathobiological cell and/or substance.

71. A method of claim 69, wherein the detection operation includes:
providing the obtained image to a trained pathobiological detection engine, the trained pathobiological detection engine being an AI/ML/DL engine trained to detect and/or identify pathobiological cells and/or substances based on a training corpus of signatures of pathobiological cells and/or substances;
receiving a positive indication from the pathobiological detection engine that the obtained image includes a pathobiological cell and/or substance therein and/or identity of that pathobiological cell and/or substance.

72. A method of claim 69, wherein detection operation includes:
providing the obtained image to a trained pathobiological detection engine, the trained pathobiological detection engine being an AI/ML/DL engine trained to detect and/or identify pathobiological cells and/or substances based on a training corpus of signatures of pathobiological cells and/or substances;
receiving a positive indication from the pathobiological detection engine that the obtained image includes a pathobiological cell and/or substance therein and/or identity of that pathobiological cell and/or substance,
wherein the obtained image includes data captured from the visible and/or non-visible electromagnetic spectrum of the scene.

73. A non-transitory computer-readable storage medium comprising instructions that when executed cause a processor of a computing device to perform operations comprising:
obtaining an image of a scene that includes in situ biological cells and/or substances therein, wherein the obtained image is micrographic, spectrographic, and/or digital;
analyzing the obtained image and detecting pathobiological cells and/or substance amongst the in-scene biological cells and/or substances;
reporting a detection the pathobiological cells and/or substances in the obtained image.

74. A non-transitory computer-readable storage medium of claim 73, wherein the operations further comprise:
sensing an environmental factor associated with the in-scene biological cells and/or substances or the environment surrounding the in-scene biological cells and/or substances;
associating the obtained environmental factor with the obtained image and/or with the detected pathobiological cell and/or substance.

75. A non-transitory computer-readable storage medium of claim 73, wherein the detection operation includes:
providing the obtained image to a trained pathobiological detection engine, the trained pathobiological detection engine being an AI/ML/DL engine trained to detect and/or identify pathobiological cells and/or substances based on a training corpus of signatures of pathobiological cells and/or substances;
receiving a positive indication from the pathobiological detection engine that the obtained image includes a pathobiological cell and/or substance therein and/or identity of that pathobiological cell and/or substance.

76. A non-transitory computer-readable storage medium of claim 73, wherein the detection operation includes:
providing the obtained image to a trained pathobiological detection engine, the trained pathobiological detection engine being an AI/ML/DL engine trained to detect and/or identify pathobiological cells and/or substances based on a training corpus of signatures of biological cells and/or substances;
receiving a positive indication from the pathobiological detection engine that the obtained image includes a pathobiological cell and/or substance therein and/or identity of that pathobiological cell and/or substance,
wherein the obtained image includes data captured from the visible and/or non-visible electromagnetic spectrum of the scene.

77. An electronic device comprising:
a scene-capture system to obtain an image of a scene that includes biological cells and/or substances therein;
a biologic identification system to analyze the obtained image and identify one or more biological cells and/or substance amongst the in-scene biological cells and/or substances;
a report system to report the identified the one or more biological cells and/or substances in the obtained image.

78. An electronic device of claim 77, wherein the identified biological cells and/or substances is a member of a type or class of biological cells and/or substances that are pathobiological.

79. An electronic device of claim 78, wherein the pathobiological biological cells includes:
pathologic cells;
diseased cells;
cancer cells;
infectious agents;
pathogens;
bioagents;
disease-producing agents; or
combination thereof.

80. An electronic device of claim 77, wherein the in-scene biological cells and/or substances are in situ.

81. An electronic device of claim 77, wherein the obtained image is micrographic, spectrographic, and/or digital.

82. An electronic device of claim 81, wherein the obtained image is micrographic because the image of the scene is captured by the scene-capture system at least in part:
on a microscopic scale;
using a microscope like magnification;
includes microscopic structures and features;
includes structures and features that are not visible to a naked human eye; or
a combination thereof.

83. An electronic device of claim 81, wherein the obtained image is spectrographic at least in part because the scene-capture system captures the image of the scene using some portion of the electromagnetic spectrum as it interacts with matter, such interactions include absorption, emission, scattering, reflection, and/or refraction.

84. An electronic device of claim 77, wherein the obtained image is hyperspectral and/or plenoptic.

85. An electronic device of claim 77, wherein the in-scene biological cells and/or substances include biological cells and/or substances that are characterized as:
physically located on a surface;
physically located in a medium;
undisturbed in their environment;
undisturbed and unadulterated;
physically located on a surface in a manner that is undisturbed and unadulterated;
not relocated for the purpose of image capture;
unmanipulated for the purpose of image capture; or
on a surface that is unaffected by the scene-capture system.

86. An electronic device of claim 77, wherein the biological cells are characterized as:
cells of a multicell biological organism;
cells of a tissue or organ of a multicell biological organism;
cells of a tumor or growth multicell biological organism;
single-celled organism;
microbes;
microscopic organisms;
single-celled organism;
living things that are too small to be seen with a human's naked eye;
a biological creature that can only be seen by a human with mechanical magnification;
microscopic spores; or
a combination thereof.

87. An electronic device of claim 77, wherein the biological cells are characterized as microbes that are characterized as:
single-celled organisms;
bacteria;
archaea;
fungi;
mold;
protists;
viruses;
microscopic multi-celled organisms;
algae;
bioagents;
spores;
germs;
prions; or
a combination thereof.

88. An electronic device of claim 77, wherein the biological cells have a size range that is selected from a group consisting of:
10-100 nanometers (nm);
10-80 nm;
10-18 nm;
15-25 nm; and
50-150 nm.

89. An electronic device of claim 77, wherein the scene includes:
one or more surfaces on which the in-scene biological cells and/or substances inhabit;
a liquid in which the in-scene biological cells and/or substances inhabit;
a bodily fluid in which the in-scene biological cells and/or substances inhabit;
an area in which the in-scene biological cells and/or substances inhabit;
a volume in which the in-scene biological cells and/or substances inhabit;
an area or volume with its dimensions falling below 0.1 mm; or
a combination thereof.

90. An electronic device of claim 77, wherein the scene-capture system to obtain a sequence of images of the scene.

91. An electronic device of claim 77, wherein the scene-capture system is characterized as:
a camera;
a digital camera;
a still image camera;
a video camera;
a digital camera with micro-optics that optical magnification for obtaining the image;
a digital camera with micro-optics are part of the electronic device itself;
a digital camera with micro-optics are part of a smartdevice with its own processor and camera;
a digital camera with micro-optics are part of additional camera functionality provided by an accessory or case for a smartdevice that operatively attaches to the smartdevice and adds the additional processing capabilities and functionalities for its scene-capture system;
a flush mounted bead lens to perform optical magnification for its scene-capture system;
a 12 or greater megapixel camera for its scene-capture system;
a 12 or greater megapixel camera with flush mounted micro-optic bead lens to do optical magnification for its scene-capture system;
a sapphire lens for its scene-capture system;
captures an image using at least some portion of the visible electromagnetic spectrum;
captures an image using at least some portion of the non-visible electromagnetic spectrum;
captures an image using infrared light waves;
captures an image using ultraviolet light waves;
captures an image using ambient in-environment electromagnetic sources;
captures images using ambient in-environment electromagnetic sources only;
captures an image using artificial visible lighting which is added to the environment via artificial lighting sources associated with the scene-capture system;
captures an image using artificial non-visible lighting which is added to the environment via artificial non-visible lighting sources associated with the scene-capture system;
captures the image of the scene with optical magnification;
captures the image of the scene with an optical magnification of a range of 20-200×;
captures the image of the scene with an optical magnification of a range of 200-1000×;
captures the image of the scene with an optical magnification of a range of 1000-5000×;
captures the image of the scene with an optical magnification of a range of 2500-5000×;
captures the image of the scene with an optical magnification of a range of 5000-10000×;
captures the image of the scene with an optical magnification of greater than 1000×;
captures the image of the scene with an optical magnification of greater than 5000×;
captures a sequence of magnified images of the scene;
enhances the image of the scene with digital magnification;
enhances the image of the scene with digital image enhancement;
enhances the image of the scene to a magnification level achievable by an electron scanning microscope (ESM); or
a combination thereof.

92. An electronic device of claim 77 further comprising an environmental sensor system to obtain an environmental factor associated with the in-scene biological cells and/or substances or the environment surrounding the in-scene biological cells and/or substances.

93. An electronic device of claim 92, wherein the obtained environmental factors includes biotic, abiotic, and associated factors.

94. An electronic device of claim 92, wherein the environmental sensor system obtains the environmental factor by measurement or sensing.

95. An electronic device of claim 92, wherein the report system to associate the obtained environmental factor with the obtained image and/or with the detected type or class of biological cell and/or substance.

96. An electronic device of claim 92, wherein the obtained environmental factors are selected from a group consisting of:
temperature;
timestamp;
humidity;
barometric pressure;
ambient sound;
location;
ambient electromagnetic activity;
ambient lighting conditions;
WiFi fingerprint;
GPS location;
airborne particle counter;
chemical detection;
gases;
radiation;
air quality;
airborne particulate matter;
atmospheric pressure;
altitude;
Geiger counter;
proximity detection;
magnetic sensor;
rain gauge;
seismometer;
airflow;
motion detection;
ionization detection;
gravity measurement;
photoelectric sensor;
piezo capacitive sensor;
capacitance sensor;
tilt sensor;
angular momentum sensor;
water-level detection;
flame detector;
smoke detector;
force gauge;
ambient electromagnetic sources;
RFID detection;
barcode reading; or
a combination thereof.

97. An electronic device of claim 77, wherein the identification of the biologic system includes operations to:
access a database of signatures of biological cells and/or substances;
isolate a biological cell and/or substance in the obtained image;
correlate the isolated biological cell and/or substance to at least one signature in the database;
determine that the correlation is significant enough to indicate a sufficient degree of confidence to identify the isolated biological cell and/or substance as being a biological cell and/or substance;
in response to that correlation determination, label the isolated biological cell and/or substance as being the determined biological cell and/or substance.

98. An electronic device of claim 77, wherein the identification of the biologic detection system includes operations to:
provide the obtained image to a trained biological identification engine, the trained biological identification engine being an AI/ML/DL engine trained to detect and/or identify biological cells and/or substances based on a training corpus of signatures of biological cells and/or substances;
receive a positive indication from the biological identification engine that the obtained image includes a biological cell and/or substance therein and/or identity of that biological cell and/or substance.

99. An electronic device of claim 77, wherein the identification of the biologic detection system includes operations to:
provide the obtained image to a trained biological identification engine, the trained biological identification engine being an AI/ML/DL engine trained to detect and/or identify biological cells and/or substances based on a training corpus of signatures of biological cells and/or substances;
receive a positive indication from the biological identification engine that the obtained image includes a biological cell and/or substance therein and/or identity of that biological cell and/or substance,
wherein the obtained image includes data captured from the visible and/or non-visible electromagnetic spectrum of the scene.

100. An electronic device of claim 77, wherein the report system to report an identification of the biological cell and/or substance in the obtained image.

101. An electronic device of claim 77, wherein the report of the report system is characterized by performing operations that:
send a communication to a human or machine that is designated to receive such communications via wired or wireless communications mechanisms;
send a notification to a human or machine that is designated to receive such notification via wired or wireless communications mechanisms;
update a database designated to receive such updates via wired or wireless communications mechanisms;
store the detection in a memory; or
a combination thereof.

102. An electronic device of claim 77, wherein the report of the report system indicates that the type of biological cells and/or substances in the obtained image is a category flagged for further research and inquiry.

103. An electronic device of claim 77 further comprising an amelioration system to react to the detection and/or identification in a manner that ameliorates the neutralizes harmful effects of the detected and/or identified biological cells and/or substances.

104. An electronic device of claim 77 further comprising an amelioration system to react to the detection and/or identification in a manner that ameliorates the neutralizes harmful effects of the detected and/or identified biological cells and/or substances, wherein the amelioration action is characterized as:
introducing an active material to a physical location of the biological cell and/or substance to neutralize the biological nature of the detected/identified biological cell and/or substance;
dispatch or request a visit by a robot or human to a physical location of the biological cell and/or substance to neutralize the biological nature of the detected/identified biological cell and/or substance;
dispatch or request a visit by a robot or human to a physical location of the biological cell and/or substance to document the conditions around the biological cell and/or substance;
activate an operation of a proximate electronic device or system that is proximate a physical location of the biological cell and/or substance to neutralize the biological nature of the detected/identified biological cell and/or substance;
activate an operation of a proximate camera that is proximate a physical location of the biological cell and/or substance to document the area around the biological cell and/or substance; or
a combination thereof.

105. A method comprising:
obtaining an image of a scene that includes biological cells and/or substances therein, wherein the obtained image is micrographic, spectrographic, and/or digital;
analyzing the obtained image and identifying one or more biological cells and/or substance amongst the in-scene biological cells and/or substances;
reporting the one or more identified biological cells and/or substances in the obtained image.

106. A method of claim 105, wherein the one or more identified biological cells and/or substances is a pathobiological.

107. A method of claim 105, wherein the in-scene biological cells and/or substances are in situ.

108. A method of claim 105 further comprising:
sensing an environmental factor associated with the in-scene biological cells and/or substances or the environment surrounding the in-scene biological cells and/or substances;
associating the obtained environmental factor with the obtained image and/or with the one or more identified biological cells and/or substances.

109. A method of claim 105, wherein the detection operation includes:
providing the obtained image to a trained biological identification engine, the trained biological identification engine being an AI/ML/DL engine trained to detect and/or identify biological cells and/or substances based on a training corpus of signatures of biological cells and/or substances;
receiving a positive indication from the biological identification engine that the obtained image includes a biological cell and/or substance therein and/or identity of that biological cell and/or substance.

110. A method of claim 105, wherein detection operation includes:
providing the obtained image to a trained biological idenfication engine, the trained biological identification engine being an AI/ML/DL engine trained to detect and/or identify biological cells and/or substances based on a training corpus of signatures of biological cells and/or substances;
receiving a positive indication from the biological identification engine that the obtained image includes a biological cell and/or substance therein and/or identity of that biological cell and/or substance,
wherein the obtained image includes data captured from the visible and/or non-visible electromagnetic spectrum of the scene.

111. A non-transitory computer-readable storage medium comprising instructions that when executed cause a processor of a computing device to perform operations comprising:
obtaining an image of a scene that includes biological cells and/or substances therein, wherein the obtained image is micrographic, spectrographic, and/or digital;
analyzing the obtained image and identifying one or more biological cells and/or substance amongst the in-scene biological cells and/or substances;
reporting the one or more identified biological cells and/or substances in the obtained image.

112. A non-transitory computer-readable storage medium of claim 111, wherein the one or more identified biological cells and/or substances is a pathobiological.

113. A non-transitory computer-readable storage medium of claim 111, wherein the in-scene biological cells and/or substances are in situ.

114. A non-transitory computer-readable storage medium of claim 111, wherein the operations further comprise:
sensing an environmental factor associated with the in-scene biological cells and/or substances or the environment surrounding the in-scene biological cells and/or substances;
associating the obtained environmental factor with the obtained image and/or with the one or more identified biological cells and/or substances.

115. A non-transitory computer-readable storage medium of claim 111, wherein the detection operation includes:
providing the obtained image to a trained biological identification engine, the trained biological identification engine being an AI/ML/DL engine trained to detect and/or identify biological cells and/or substances based on a training corpus of signatures of biological cells and/or substances;
receiving a positive indication from the biological identification engine that the obtained image includes a biological cell and/or substance therein and/or identity of that biological cell and/or substance.

116. A non-transitory computer-readable storage medium of claim 111, wherein the detection operation includes:
providing the obtained image to a trained biological identification engine, the trained biological identification engine being an AI/ML/DL engine trained to detect and/or identify biological cells and/or substances based on a training corpus of signatures of biological cells and/or substances;
receiving a positive indication from the biological identification engine that the obtained image includes a biological cell and/or substance therein and/or identity of that biological cell and/or substance,
wherein the obtained image includes data captured from the visible and/or non-visible electromagnetic spectrum of the scene.
